(19) United States
(12) Patent Application Publication
(10) Pub. No.: US 2014/0314790 A1
(43) Pub. Date: Oct. 23, 2014

(54) CASPASE 9 INHIBITION AND BRI2 PEPTIDES FOR TREATING DEMENTIA

(71) Applicant: ALBERT EINSTEIN COLLEGE OF MEDICINE OF YESHIVA UNIVERSITY, Bronx, NY (US)

(72) Inventors: Luciano D'Adamio, New York, NY (US); Robert Tamayev, Forest Hills, NY (US); Shuji Matsuda, Bronx, NY (US)

(21) Appl. No.: 14/347,129

(22) PCT Filed: Oct. 2, 2012
(86) PCT No.: PCT/US12/58480

§ 371 Date: Mar. 25, 2014

Related U.S. Application Data
(60) Provisional application No. 61/542,937, filed on Oct. 4, 2011.
(60) Provisional application No. 61/645,676, filed on May 11, 2012.

(51) Int. Cl. A61K 38/17; C12N 15/113; C07K 16/40

(52) U.S. Cl. CPC A61K 38/17; C07K 16/40; C12N 15/1137
USPC 424/158.1; 514/17.7; 514/44 A

(57) ABSTRACT

Methods are provided for treating dementia and or impaired cognition, as well as assays useful for identifying novel anti-dementia agents. Compounds and compositions for treating dementia and or impaired cognition are also provided.

### CROSS-REFERENCE TO RELATED APPLICATIONS

This application claims benefit of U.S. Provisional Application No. 61/542,937, filed Oct. 4, 2011, and of U.S. Provisional Application No. 61/645,676, filed May 11, 2012, the contents of each which are hereby incorporated by reference.

### STATEMENT OF GOVERNMENT SUPPORT

This invention was made with government support under grant number R01 AG033007 awarded by the National Institutes of Health. The government has certain rights in the invention.

### BACKGROUND OF THE INVENTION

Throughout this application various publications are referred to in parentheses by author and year. Full citations for these references may be found at the end of the specification. The disclosures of each of these publications, and of all patents, patent application publications and books cited herein, are hereby incorporated by reference in their entirety into the subject application to more fully describe the art to which the subject invention pertains.

Familial dementias, which include Alzheimer disease (AD), familial British dementia (FBD), and familial Danish dementia (FDD), are caused by dominantly inherited autosomal mutations and are characterized by the production of amyloidogenic peptides, neurofibrillary tangles (NFTs) and neurodegeneration (Cole & Vassar, 2007; De Strooper et al, 2010; Bertram et al, 2010; St George-Hyslop & Petit, 2005; Vidal et al, 2000; Hardy & Selkoe, 2002). The prevailing pathogenic theory for such dementias, the “amyloid cascade hypothesis” (Hardy & Selkoe, 2002), posits that the accumulation of amyloidogenic peptides triggers tauopathy, neurodegeneration, and cognitive and behavioral changes. However, this hypothesis is yet to be validated, and causes of dementia may be multifaceted and involve other mechanisms, such as loss of function due to pathogenic mutations.

The present invention address the need for targeted anti-dementia treatments and therapies for arrest or reduction of cognitive impairment and provides novel assays for identifying therapeutic agents.

### SUMMARY OF THE INVENTION

A method is provided of treating a dementia and/or an impaired cognition in a subject comprising administering to the subject an amount of an inhibitor of caspase-9, of caspase-6 or of caspase-8 sufficient to treat dementia and/or impaired cognition.

A method is also provided of treating a dementia and/or impaired cognition in a subject comprising administering to the subject an amount of an agent comprising an active fragment of a BRI2 peptide or an active analog of a fragment of a BRI2 peptide sufficient to treat dementia and/or impaired cognition.

A method is also provided of treating a dementia and/or impaired cognition in a subject comprising administering to the subject an amount of an inhibitor of amino terminal soluble APPβ (sAPPβ) sufficient to treat dementia and/or impaired cognition.

A method is also provided for identifying an agent for treating dementia and/or impaired cognition in a subject comprising contacting an amyloid precursor protein (APP) with the agent in the presence of a secretase and comparing the production of sAPPβ by the secretase in the presence of the agent and in the absence of the agent, wherein inhibition of production of sAPPβ by the agent indicates the agent as suitable for treating dementia and/or impaired cognition.

A method is provided for identifying an agent for treating dementia and/or impaired cognition in a subject comprising contacting an amino terminal soluble APPβ (sAPPβ) with the agent and comparing activity of the sAPPβ in the presence and in the absence of the agent, wherein inhibition by the agent of the sAPPβ indicates the agent as suitable for treating dementia and/or impaired cognition.

A method is also provided for identifying an agent for treating dementia and/or impaired cognition in a subject comprising contacting a caspase-9, caspase-6 or caspase-8 with the agent and comparing activity of the caspase-9, caspase-6 or caspase-8 in the presence and in the absence of the agent, wherein inhibition by the agent of the caspase-9, caspase-6 or caspase-8 indicates the agent as suitable for treating dementia and/or impaired cognition.

A method is also provided for identifying an agent for treating dementia and/or impaired cognition in a subject comprising contacting an amino terminal soluble APPβ (sAPPβ) with the agent and comparing activity of the sAPPβ in the presence and in the absence of the agent, and contacting a caspase-9, caspase-6 or caspase-8 with the agent and comparing activity of the caspase-9, caspase-6 or caspase-8 in the presence and in the absence of the agent, wherein inhibition by the agent of both the caspase-9, caspase-6 or caspase-8 and the sAPPβ indicates the agent as suitable for treating dementia and/or impaired cognition.

An inhibitor of caspase-9, caspase-6 or caspase-8 is provided, or an inhibitor of sAPPβ, or an inhibitor of production of sAPPβ, for treating dementia or impaired-cognition in a subject.

An inhibitor of caspase-9 is provided comprising DXVYYCGLKY (SEQ ID NO:10) or ADVYYCGLKY (SEQ ID NO:12) or DDVYYCGLKYIKDD (SEQ ID NO:9).

A composition is provided comprising an inhibitor of caspase-9 as described hereinabove and a pharmaceutically acceptable carrier.

Also provided is a method of identifying a small molecule that inhibits APP processing comprising a) modeling in silico (i) the 3-dimensional site or sites on SEQ ID NO:4, 5, 9, 10 or 12 that bind APP, or (ii) the 3-dimensional site or sites on APP which bind SEQ ID NO:4, 5, 9, 10 or 12; b) testing in silico if the small molecule (i) binds to the modeled 3-dimensional site on APP or (ii) mimics the 3-dimensional site or sites on SEQ ID NO:4, 5, 9, 10 or 12 that bind APP; and c) determining in vitro if the small molecule identified as (i) binding to the site or sites in silico or (ii) mimicking the 3-dimensional site or sites on SEQ ID NO:4, 5, 9, 10 or 12 in b), binds to APP and inhibits its processing.

Also provided is a method of identifying a small molecule that inhibits APP processing comprising determining in vitro if a small molecule identified as (i) binding to the site or sites of APP previously determined to be bound by SEQ ID NO:4, 5, 9, 10 or 12, or (ii) mimicking the 3-dimensional site or sites on SEQ ID NO:4, 5, 9, 10 or 12 that bind APP, binds to APP and inhibits its processing.

### BRIEF DESCRIPTION OF THE DRAWINGS

FIG. 1A-1B: Caspase 9 inhibition but not caspase 3 inhibition reverses LTP defects in FDDKI mice. (A) Active caspase-9 fragment, but no active caspase-3, is present in FDDKI mice synaptosomes. APP, mTor and NR2a signals show that similar amounts of proteins were analyzed. (B) Sixty minute perfusion with caspase 9 inhibitor Z-LEHD-FMK, 2 μM [F(1,10)=20.222 P=0.001] (SEQ ID NO:13), and general caspase Z-VAD-FMK, 10 μM [F(1,12)=10.787 P=0.007], but not the caspase 3 inhibitor Z-DEVD-FMK (SEQ ID NO:14), 2 μM [F(1, 10)=1.681, P=0.231], reverse LTP impairment in FDDKI mice. Z-LEHD-FMK does not alter LTP of WT mice [WT untreated vs. WT treated F(1, 6)=0.173. P=0.692].

FIG. 2A-2B. Inhibiting caspase 9 but not caspase 3 rescue the memory deficit of FDDKI mice. Mice were injected in the lateral ventricle with either 1 μl of PBS/500 μM caspase-9 inhibitor or 1 μl of PBS/500 μM caspase-3 inhibitor. Injections were performed 1 hr prior to the training section and, the following day, 1 hr before testing. (A) WT and FDDKI mice spent the same amount of time exploring the two identical objects on day 1. As the mice develop habituation to the test, they tend to explore the objects more. (B) WT mice spent more time exploring the novel object 24 hours later, showing normal object recognition (discriminatory ratio=0.63), while FDDKI mice present amnesia and do not distinguish the new object from the old one (discriminatory ratio=0.5). 1 μl of PBS/500 μM caspase-9 inhibitor transiently rescue this memory deficit, while 1 μl of PBS/500 μM caspase-3 inhibitor does not. The number of days between the day 2 of a test and day 1 of the following test are indicated (x d.).

FIG. 3A-3G. Increased levels of cleaved caspase-3 and -6 in synaptosomal fractions of FDDKI mice. A, P2 represents the synaptosomal fraction (enriched in the post-synaptic density protein PSD95), while the S2 fraction is enriched in soluble cytosolic proteins (such as tau). Interestingly, APP and APP-CTFs are slightly enriched in synaptic preparations as compared to the S1 (post nuclear supernatant) fraction. Ten μg of protein were loaded in each lane. B, Western blot analysis of caspase-3 and caspase-6 (for cl.-caspase-6 a longer exposure is also shown) in total homogenates (S1) and hippocampal synaptic fractions (P2) from 12 months old mice (samples from 3 mice for each genotype are shown). Cl-caspase-6 is found in the synaptic fraction of both FDDKI and WT mice. Cl.-caspase-6 levels are increased in FDDKI mice. Cl.-caspase-3 is only detectable in FDDKI mice, and is found in both S1 and P2 fractions (albeit it is enriched in P2 fractions). (C) Quantification of the data shown in B indicates that synaptic fractions from Danish mice express significantly more cl.-caspase-6 (P=.0.012) than WT littermates. Error bars indicate s.e.m.

FIG. 4A-4C. High levels of active initiator caspase-9 in FDDKI mice. A, Homogenates (input) were prepared from the bVAD injected (+bVAD) and contralateral non-injected (con.) hippocampal regions of WT and Danish mice. Active caspases were isolated from homogenates with streptavidin-agarose-beads pull-down. Western blot analysis shows that the caspase inhibitor bVAD traps FL-caspase-9 only from the bVAD injected FDDKI mouse hippocampus; FL-caspase-8, cl.caspase-3 and cl.-caspase-6 are not trapped. B, In a similar experiment, the streptavidin-agarose-beads pull-down experiment was performed from the P2 fractions. Again, active FL-caspase-9 is isolated from FDDKI but not WT mice. C, Organotypic hippocampal cultures from either FDDKI or WT mice were incubated for 3 hrs with 45 μM bVAD. After lysis, active caspases were isolated from homogenates. Again, caspase-9 was the only active caspase isolated. Albeit traces of active caspase-9 are found in the WT samples, the levels found in the FDDKI hippocampus are greatly elevated. The blots shown in A, B and C are representative of duplicate experiments.

FIG. 5A-5D. Specific inhibition of active caspase-9 with Pen1-XBIR3 rescues the memory deficits of FDDKI mice. A cannula was surgically implanted in the lateral ventricle of a cohort of 6-month-old FDDKI mice and WT littermates. Twenty-five days after surgery mice were injected in the lateral ventricle either with 2 μl of PBS/23 μM Pen1-XBIR3, 2 μl of PBS/16 μM Pen1-CrmA, or 2 μl of PBS alone (WT/PBS N=7, WT/Pen1-XBIR3 N=8, WT/Pen1-CrmA N=7, FDDKI/PBS N=8, FDDKI/Pen1-XBIR3 N=8, FDDKI/Pen1-CrmA N=8). Injections were performed 1 h prior to the training section and 1 h before testing on the following day. WT and FDDKI mice spent the same amount of time exploring the two identical objects on day 1 (A). WT mice spent more time exploring the novel object 24 h later, showing normal object recognition (discriminatory ratio 0.63), while FDDKI mice present amnesia (WT/Vehicle vs. FDDKI/Vehicle P=0.0007) and do not distinguish the new object from the old one (discriminatory ratio 0.5). Pen1-XBIR3 rescues this memory deficit (FDDKI/Pen1-XBIR3 vs. WT/Vehicle P=0.79; FDDKI/Pen1-XBIR3 vs. WT/Pen1-XBIR3 P=0.89; FDDKI/Pen1-XBIR3 vs. WT/Pen1-CrmA P=0.37; FDDKI/Pen1-XBIR3 vs. FDDKI/Vehicle P=0.0013; FDDKI/Pen1-XBIR3 vs. FDDKI/Pen1-CrmA P=0.0027), while Pen1-CrmA does not (FDDKI/Pen1-CrmA vs. WT/Vehicle P=0.0079; FDDKI/Pen1-CrmA vs. WT/Pen1-XBIR3 P=0.0038; FDDKI/Pen1-CrmA vs. WT/Pen1-CrmA P=0.034; FDDKI/Pen1-CrmA vs. FDDKI/Vehicle P=0.24). (B). C and D, The NOR test was repeated 5 days later without further treatments. The therapeutic effect of Pen1-XBIR3 is still significant (WT/Vehicle vs. FDDKI/Vehicle P=0.0003; FDDKI/Pen1-XBIR3 vs. WT/Vehicle P=0.046; FDDKI/Pen1-XBIR3 vs. WT/Pen1-XBIR3 P=0.44; FDDKI/Pen1-XBIR3 vs. WT/Pen1-CrmA P=0.95; FDDKI/Pen1-XBIR3 vs. FDDKI/Vehicle P=0.03; FDDKI/Pen1-XBIR3 vs. FDDKI/Pen1-CrmA P=0.028; FDDKI/Pen1-CrmA vs. WT/Vehicle P=0.0002; FDDKI/Pen1-CrmA vs. WT/Pen1-XBIR3 P=0.0025; FDDKI/Pen1-CrmA vs. WT/Pen1-CrmA P=0.0038; FDDKI/Pen1-crmA vs. FDDKI/Vehicle P=0.85).

FIG. 6A-6D. Model depicting the mechanisms by which caspase-9 can lead to alteration typical of neurodegenerative disorders: memory loss, dystrophic neurites and neuronal loss. A and B, Due to loss of BRI2 protein (loss of function model), APP processing is increased during synaptic transmission and memory acquisition in FDD leading to increased production of sAPPβ and β-CTF. Caspase-9 is activated via and unknown mechanisms by β-CTF and/or sAPPβ, perhaps via interaction with a membrane-bound receptor, sAPPβ-R, such as DR6 (Nikolaev et al, 2009). This increased caspase-9 activation leads to memory deficits via a yet to be defined mechanism. Whether sAPPα and/or α-CTF can also trigger this pathway remains to be determined. In this context, it is worth noting that BRI2 also inhibits α-secretase processing of APP (Matsuda et al, 2005; Matsuda et al, 2008; Tamayev et al, 2011b). Further studies will be needed to assess the role of the α-processing pathway of APP in dementia. C, Caspase-9 is activated by a pathway that is dependent on the Danish mutation but independent of β-CTF and/or sAPPβ. Active caspase-9 and β-CTF and/or sAPPβ activate two distinct noxious pathways that are necessary but not sufficient to prompt synaptic/memory deficits. D, Aberrant activation of caspase-9 in synaptosomes can initially cause functional impairments leading to synaptic plasticity and memory acquisition deficits, with no noticeable anatomical changes. Repetitive cycles of high caspase-9 activity can lead to dystrophy of neurites. Prolonged and sustained activation of caspase-9 increases the probability that in any given neuron caspase-9 activity may leak to the cell body and prompt the demise of the neuron.

FIG. 7A-7E. Mapping the BRI2 domain that binds APP and inhibits APP processing. (A) APP is cleaved by β-secretase into sAPPβ and β-CTF. γ-cleavage of β-CTF yields Aβ and AID/AICD peptides. Alternatively, α-secretase clips APP into sAPPα and α-CTF. α-CTF is cut by γ-secretase into P3 and AID. (B-C) BRI2 binds APP and inhibits processing by α- and β-secretases. Binding of BRI2 to β-CTF inhibits cleavage by γ-secretase. (D) constructs and domains [cytoplasmic (Cyt), transmembrane (TM), extracellular (Lumen), brichos (B) and convertases-cleavage site, myc-tag]. Lysates (L) and α-myc immunoprecipitates (myc-IP) from transfected cells were analyzed by Western blot (WB) for BRI2, APP and APP-CTFs. (E) APP-Gal4, AIDGal4, Gal4-depended promoter, luciferase reporter, cytoplasm (Cyt) and nucleus (Nc) are schematically indicated. Luciferase activity is expressed as % of the activity in cells transfected with APP-Gal4, luciferase reporter and empty vector (vec).

FIG. 8A-8I. 4A BRI2-derived peptide binds APP and inhibits β-cleavage of APP. (A-B) HEK293-APP cells were incubated with the indicated peptides (N1, N2, N3, N4, N5, N6 and N8 are SEQ ID NOs: 2, 3, 4, 5, 6, 7, 8, and 9, respectively). β- and α-cleavage of APP were quantified by measuring sAPPβ and sAPPα in media by WB. WB of cell lysates detected APP and α-Tubulin. (C-E) WB analysis of lysates (L) or α-Flag IP (IP) from HeLa/APP cells incubated for 2 hours with Flagged peptides. (C) Cells were incubated at either 37° C. or 40° C. with or without 40 μM N3-2A-F. (D) The indicated concentrations of N3-2A (SEQ ID NO:3) was added to the media containing 40 μM N3-2A-F. (E) cells were incubated with 40 μM N3-2A-F, N4-F or N3-4A-F. (F) Brain cells were cultured as in (C). (G) Biotinylated cells were cultured as in (C). The reduced and not reduced samples are indicated (+red and −red, respectively). Lysates (L), α-Flag IP eluted with Flag-peptide (E), eluted sample precipitated with streptavidin-beads [both the fraction unbound (U) and bound (B) to streptavidin-beads], were probed for APP in WB. (H) Purified β-secretase was incubated with fluorescent β-secretase substrate for 30 minutes, resulting in β-cleavage that could be detected by fluorescence increase. In separate samples, the indicated concentrations of N3-2A or β-secretase-inhibitor IV were added to the reaction. The data are shown as % of inhibition of β-secretase activity in samples without inhibitors. (I) Model of N3-2A/MoBA activity. The peptide interferes with processing of APP by β-secretase but, unlike full-length BRI2, does not modulate γ-cleavage of β-CTF.

FIG. 9A-9C. MoBA and a β-secretase inhibitor rescue the LTP deficit of FDDKI mice—a γ-secretase inhibitor (GSI) does not. (A) Sixty-minutes perfusion with MoBA reverses LTP impairment in FDDKI mice [WT to FDDKI: F(1,12)=12.372, P=0.004; WT to FDDKI+MoBA 1 μM: F(1,12)=0.012, P=0.914; WT to FDDKI+MoBA 10 nM: F(1,11)=0.202, P=0.662; FDDKI to FDDKI+MoBA 1 μM: F(1,12)=(10.078), P=0.006; FDDKI to FDDKI+MoBA 10 nM: F(1,11)=15.049, P=0.008]. N6 does not rescue the LTP deficit [FDDKI to FDDKI+N6 1 μM: F(1,10)=0.053, P=0.821]. MoBA does not alter LTP of WT mice [WT to WT+MoBA 1 μM: F(1,12)=0.361, P=0.560]. (B) β-secretase inhibitor IV (50 nM; IC₅₀=15 nM) rescues LTP impairment in FDDKI mice [FDDKI to FDDKI+β-secretase-inhibitor IV: F(1,14)=12.258, P=0.004; WT to FDDKI: F(1,13)=12.272, P=0.004; WT to FDDKI+β-secretase-inhibitor IV: F(1,13)=0.604, P=0.451]. There was a trend toward increased LTP in inhibitor IV-treated WT and FDDKI samples versus vehicle-treated WT controls, but this difference was not statistically significant. Compound-E (1 nM; IC₅₀=300/240 pM) does not rescue the LTP defect in FDDKI samples [FDDKI to FDDKI+compound-E: F(1,11)=0.838, P=0.380]. The β- and γ-secretase inhibitors do not alter LTP of WT mice [WT to WT+β-secretase-inhibitor IV: F(1,10)=0.413, P=0.535; WT to WT+compound-E: F(1,11)=0.041, P=0.844]. (C) Lysates from hippocampal slices treated with (+) or without (−) compound-E for 3 hours were analyzed by WB for APP and CTFs. The bottom graph represents quantization of triplicate samples. The CTFs levels are expressed as a % of APP.

FIG. 10A-10C Inhibiting 3-cleavage of APP rescue the memory deficit of FDDKI mice. Mice were injected in the lateral ventricle with either 1 μl of PBS/100 μM β-secretase-inhibitor IV, 1 μl of PBS/300 nM compound-E, 1 μl of PBS/100 μM-MoBA or 1 μl of PBS/3 μM compound-E. Injections were performed 1 hr prior to the training section and, the following day, 1 hr before testing. (A) WT and FDDKI mice spent the same amount of time exploring the two identical objects on day 1. As the mice develop habituation to the test, they tend to explore the objects more. (B) WT mice spent more time exploring the novel object 24 hours later, showing normal object recognition (discriminatory ratio=0.63), while FDDKI mice present amnesia and do not distinguish the new object from the old one (discriminatory ratio=0.5). β-secretase-inhibitor IV and MoBA transiently rescue this memory deficit, while GSI does not. The number of days between the day 2 of a test and day 1 of the following test are indicated (x d.). (C) Model depicting early pathogenic events preceding amyloidosis and leading to memory loss. Two inhibitors of β-cleavage of APP (Inhibitor IV and MoBA), but not a GSI, rescue the LTP/memory deficits, suggesting that newly synthesized sAPPβ and/or β-CTF, but not Aβ/P3/AID cause these deficits in FDDKI mice (+ and in black).

### DETAILED DESCRIPTION OF THE INVENTION

### Abbreviations

AD—Alzheimer's disease
APP—amyloid precursor protein
WB—Western blot
CTF—C-terminal fragment
GSI—γ-secretase inhibitor
sAPPβ—soluble amyloid precursor protein-beta
LTP—long-term potentiation
MoBA—modulator of β-cleavage of APP
PBS—phosphate buffered saline
FDDKI—Familial Danish Dementia knock-in
FBD—Familial British Dementia
FDD—Familial Danish Dementia

A method is provided of treating a dementia and/or an impaired cognition in a subject comprising administering to the subject an amount of an inhibitor of caspase-9, of caspase-6 or of caspase-8 sufficient to treat dementia and/or impaired cognition.

A method is also provided of treating a dementia and/or impaired cognition in a subject comprising administering to the subject an amount of an agent comprising an active fragment of a BRI2 peptide or an active analog of a fragment of a BRI2 peptide sufficient to treat dementia and/or impaired cognition.

A method is also provided of treating a dementia and/or impaired cognition in a subject comprising administering to the subject an amount of an inhibitor of amino terminal soluble APPβ (sAPPβ) sufficient to treat dementia and/or impaired cognition.

With regard to the above-described methods, in an embodiment, the method is for treating a dementia and the dementia is a familial dementia or is caused by Alzheimer's disease.

In an embodiment, the inhibitor of caspase-9, caspase-6 or caspase-8 or the agent is administered to the subject in a manner effective to cross a central nervous system blood-brain barrier. In an embodiment, the inhibitor of caspase-9, caspase-6 or caspase-8 or the agent is administered systemically to the subject. In an embodiment, the inhibitor of caspase-9, caspase-6 or caspase-8 or the agent is administered into the central nervous system of the subject. In an embodiment, the inhibitor of caspase-9, caspase-6 or caspase-8 or the agent is administered into a cerebral ventricle of the subject. In an embodiment, the cerebral ventricle is a lateral ventricle. In an embodiment, the inhibitor of caspase-9, caspase-6 or caspase-8 or the agent is administered via an implant in the subject. In an embodiment, the implant is an implanted catheter or pump. In an embodiment, the implant is implanted into the central nervous system of the subject. In an embodiment, the inhibitor of caspase-9, caspase-6 or caspase-8 or the agent is administered continuously to the subject.

In an embodiment, the subject is administered the inhibitor of caspase-9 and the inhibitor of caspase-9 is z-LEHD-fmk. In an embodiment, the subject is administered an active fragment of a BRI2 peptide, and the BRI2 peptide comprises consecutive amino acid residues having the sequence set forth in SEQ ID NO:1. In an embodiment, the subject is administered an agent comprising SEQ ID NO:4, SEQ ID NO:5, SEQ ID NO:9, SEQ ID NO:10 or SEQ ID NO:12.

In an embodiment, the inhibitor is an inhibitor of caspase-9 and comprises XIAP-BIR3 domain (“XBIR3”). In an embodiment, the XBIR3 comprises SEQ ID NO:17. In an embodiment, the XBIR3 comprises SEQ ID NO:17 with 1, 2 or 3 additional amino acids at the N-terminus, C-terminus, or independently, at both termini.

In an embodiment, the inhibitor is an inhibitor of caspase-9 and is XIAP-BIR3 domain disulfide-linked to a cell-penetrating peptide. In an embodiment, the cell-penetrating peptide is Penetratin1 (“Pen1-XBIR3”). In an embodiment, the Penetratin1 comprises SEQ ID NO:18 (RQIKIWFQNRRMKWKK). In an embodiment the inhibitor of caspase-9 is SEQ ID NO:17 linked through a disulfide bond to SEQ ID NO:18. In an embodiment, SEQ ID NO:17 and SEQ ID NO:18 are in an equimolar ratio. In an embodiment the inhibitor of caspase-9 is SEQ ID NO:17 linked by a peptide bond to SEQ ID NO:18.

In an embodiment, the subject has been diagnosed or identified as suffering from the dementia or impaired cognition prior to administration of the inhibitor or agent. In an embodiment, the methods further comprise diagnosing or identifying the subject as suffering from the dementia or the impaired cognition prior to administration of the inhibitor or the agent.

In an embodiment, the subject has not suffered a stroke. In an embodiment, the method is for treating impaired cognition in the subject.

A method is also provided for identifying an agent for treating dementia and/or impaired cognition in a subject comprising contacting an amyloid precursor protein (APP) with the agent in the presence of a secretase and comparing the production of sAPPβ by the secretase in the presence of the agent and in the absence of the agent, wherein inhibition of production of sAPPβ by the agent indicates the agent as suitable for treating dementia and/or impaired cognition.

In an embodiment, the APP is a human APP. In an embodiment, the agent is a peptide or a small molecule. In an embodiment, the secretase is a β-secretase. In an embodiment, the agent does not modulate γ-cleavage of β-CTF and/or does not bind β-CTF. In an embodiment, the agent does not inhibit γ-secretase. In an embodiment, the method further comprises identifying the agent as not inhibiting γ-secretase activity. In an embodiment, the method further comprises identifying the agent as not modulating γ-cleavage of β-CTF and/or not binding β-CTF.

A method is provided for identifying an agent for treating dementia and/or impaired cognition in a subject comprising contacting an amino terminal soluble APPβ (sAPPβ) with the agent and comparing activity of the sAPPβ in the presence and in the absence of the agent, wherein inhibition by the agent of the sAPPβ indicates the agent as suitable for treating dementia and/or impaired cognition.

In an embodiment, the agent is identified as inhibiting sAPPβ if it inhibits production of sAPPβ.

A method is also provided for identifying an agent for treating dementia and/or impaired cognition in a subject comprising contacting a caspase-9, caspase-6 or caspase-8 with the agent and comparing activity of the caspase-9, caspase-6 or caspase-8 in the presence and in the absence of the agent, wherein inhibition by the agent of the caspase-9, caspase-6 or caspase-8 indicates the agent as suitable for treating dementia and/or impaired cognition.

A method is also provided for identifying an agent for treating dementia and/or impaired cognition in a subject comprising contacting an amino terminal soluble APPβ (sAPPβ) with the agent and comparing activity of the sAPPβ in the presence and in the absence of the agent, and contacting a caspase-9, caspase-6 or caspase-8 with the agent and comparing activity of the caspase-9, caspase-6 or caspase-8 in the presence and in the absence of the agent, wherein inhibition by the agent of both the caspase-9, caspase-6 or caspase-8 and the sAPPβ indicates the agent as suitable for treating dementia and/or impaired cognition.

In an embodiment, the agent is identified as inhibiting sAPPβ if it inhibits production of sAPPβ.

In an embodiment, the method further comprises determining whether the agent does not inhibit caspase-3, wherein inhibition by the agent of the caspase-9 but not the caspase-3 indicates the agent as suitable for treating dementia and/or impaired cognition

An inhibitor of caspase-9, caspase-6 or caspase-8 is provided, or an inhibitor of sAPPβ, or an inhibitor of production of sAPPβ, for treating dementia or impaired-cognition in a subject.

In an embodiment, the inhibitor of caspase-9 selectively inhibits caspase-9 and not other caspases.

An inhibitor of caspase-9 is provided comprising DXVYYCGLKY (SEQ ID NO:10) or ADVYYCGLKY (SEQ ID NO:12) or DDVYYCGLKYIKDD (SEQ ID NO:9). In an embodiment, X in SEQ ID NO:10 is D. In an embodiment, X in SEQ ID NO:10 is A.

In an embodiment, the inhibitor is an inhibitor of caspase-9 and comprises XIAP-BIR3 domain (“XBIR3”). In an embodiment, the XBIR3 comprises SEQ ID NO:17. In an embodiment, the XBIR3 comprises SEQ ID NO:17 with 1, 2 or 3 additional amino acids at the N-terminus, C-terminus, or independently, at both termini.

In an embodiment, an inhibitor of caspase-9 is provided comprising XIAP-BIR3 domain disulfide-linked to a cell-penetrating peptide. In an embodiment, the cell-penetrating peptide is Penetratin1 (“Pen1-XBIR3”). In an embodiment, the Penetratin1 comprises SEQ ID NO:18 (RQIKIWFQNRRMKWKK). In an embodiment the inhibitor of caspase-9 is SEQ ID NO:17 linked through a disulfide bond to SEQ ID NO:18. In an embodiment, SEQ ID NO:17 and SEQ ID NO:18 are in an equimolar ratio. In an embodiment the inhibitor of caspase-9 is SEQ ID NO:17 linked by a peptide bond to SEQ ID NO:18.

As used herein, a “dementia” is an art-recognized disease state mainly characterized by the impairment of cognition in a subject (but does not include a cognitive impairment caused by delirium), and is usually progressive. It affects primarily the older human population, but can occur in younger subjects also. Dementia may be due to, inter alia, Alzheimer's disease. Dementia may also be, in non-limiting examples, familial dementia, vascular dementia, Lewy body dementia, frontotemporal dementias, and HIV-associated dementia. Patients can have more than one type of dementia (mixed dementia). Dementia is distinct from normal age-associated memory impairment, which is an impairment that is not severe enough to affect daily function and where learning may be still effected if the subject is given sufficient time. Diagnostic and Statistical Manual of Mental Disorders IV-TR, ((2000), American Psychiatric Association), which is hereby incorporated by reference, provides a reference account of dementia, and of cognitive impairment, and identification/diagnosis thereof.

As used herein, “cognitive impairment” in a subject is a state of cognitive impairment beyond that expected for the age of the subject as recognized in the art.

The invention is directed to methods of treating a disease in a subject characterized by a dementia and/or cognitive impairment. In an embodiment, the dementia is due to Alzheimer's disease. In an embodiment, the dementia is a familial dementia. In an embodiment, the dementia comprises a synaptic dysfunction etiology. In an embodiment, the dementia comprises a neuronal cell death etiology.

As used herein, “treating” dementia and/or cognitive impairment means that one or more symptoms of the disease, such as the dementia or cognitive impairment itself, or other parameters by which the disease is characterized such as memory deficit, including loss of short-term memory and confusion are reduced, ameliorated, prevented, or reversed at least in part.

In an embodiment, the dementia is Alzheimer's dementia. In an embodiment, the dementia is a familial dementia. In an embodiment, the familial dementia is Familial British Dementia (FBD), which is characterized by a point mutation at the stop codon of BRI2, resulting in read-through into the 3′-untranslated region and the synthesis of a BRI2 protein containing 17 extra amino acids at the COOH-terminus. In an embodiment, the familial dementia is Familial Danish Dementia (FDD) where the presence of a 10-nucleotide duplication one codon before the normal stop codon produces a frame-shift in the BRI2 sequence generating a larger-than-normal precursor protein, of which the amyloid subunit comprises the last 34 COOH-terminal amino acids.

BRI2 peptide is disclosed in U.S. Patent Application Publication No. 2010-0098682 A1, which is hereby incorporated by reference in its entirety. In an embodiment, the agent comprises an active fragment of BRI2 peptide or an active analog of a fragment of BRI2 peptide. In an embodiment, the BRI2 peptide is human BRI2 peptide. In an embodiment, the human BRI2 peptide has the sequence set forth in SEQ ID NO:1 (GenBank Q9Y287):

| | |
|---|---|
| 1 | MVKVTFNSAL AQKEAKKDEP KSGEEALIIP |
| | PDAVAVDCKD PDDVVPVGQR RAWCWCMCFG |
| 61 | LAFMLAGVIL GGAYLYKYFA LQPDDVYYCG |
| | IKYIKDDVIL NEPSADAPAA LYQTIEENIK |
| 121 | IFEEEEVEFI SVPVPEFADS DPANIVHDFN |
| | KKLTAYLDLN LDKCYVIPLN TSIVMPPRNL |
| 181 | LELLINIKAG TYLPQSYLIH EHMVITDRIE |
| | NIDHLGFFIY RLCHDKETYK LQRRETIKGI |
| 241 | QKREASNCFA IRHFENKFAV ETLICS |

The fragment of BRI2 peptide does not comprise full length BRI2 peptide. In an embodiment the active fragment of BRI2 peptide is a 10-14 mer. In an embodiment the active fragment of BRI2 peptide is a 10-mer. In an embodiment the active fragment of BRI2 peptide is a 14-mer. In an embodiment the fragment of BRI2 peptide comprises or consists of SEQ ID NO:4 or 9. In an embodiment, the active analog of the fragment of BRI2 peptide is an active analog of a fragment of human BRI2 peptide. In an embodiment, the active analog of a fragment of BRI2 peptide is at least 90% homologous, at least 95% homologous or at least 99% homologous to a fragment of SEQ ID NO:1. In an embodiment the fragment of BRI2 peptide comprises or consist of SEQ ID NO:5 or 12. As used herein, “active” as in active analog and active fragment means possessing the ability to ameliorate a dementia and/or impaired cognition and/or possessing the ability to inhibit human caspase-9 and/or impair activity or production of soluble amyloid precursor protein beta (sAPPβ). In an embodiment the active fragment comprises the sequence DDVYYCGLKY (SEQ ID NO:4) (“N3”), DAVYYCGLKY (SEQ ID NO:5) (“N3-2A”), ADVYYCGLKY (SEQ ID NO:12) (“N3-1A”) or DDVYYCGLKYIKDD (SEQ ID NO:9) (“N8”). In an embodiment, the fragment does not comprise one of, or does not comprise any of, the following sequences: YLYKYFALQP (SEQ ID NO:2), FALQPDDVYY (SEQ ID NO:3), CGLKYIKDDV (SEQ ID NO:6), IKDDVILNEP (SEQ ID NO:7) and ILNEPSADAP (SEQ ID NO:8). In an embodiment, the fragment comprises the sequence DXVYYCGLKY (SEQ ID NO:10), wherein X is any amino acid. In an embodiment, X is D or A. In an embodiment, the fragment or active analog thereof does not modulate γ-cleavage of β-CTF.

In an embodiment, the active fragment of BRI2 peptide or active analog of a fragment of BRI2 peptide comprises a peptidomimetic, i.e. a compound that is capable of mimicking a natural parent amino acid in a protein, in that the substitution of an amino acid with the peptidomimetic does not significantly affect the activity of the protein. Peptides and proteins comprising peptidomimetics are generally poor substrates of proteases and are generally to be active in vivo for a longer period of time as compared to the natural proteins. Many non-hydrolyzable peptide bond analogs are known in the art, along with procedures for synthesis of peptides containing such bonds. Non-hydrolyzable bonds include —CH₂NH, —COCH₂,

—CH(CN)NH, —CH₂CH(OH), —CH₂O, and —CH₂S. In addition, peptidomimetic-containing peptides could be less antigenic and show an overall higher bioavailability. The skilled artisan would understand that design and synthesis of proteins comprising peptidomimetics would not require undue experimentation.

Active analogs may comprise one or more D-amino acid, retro-inverso and/or inverso substituted versions of the active peptides. Activity is routinely determinable by, for example, early-ADMET studies to compare stability. Serum stability and serum binding in both mammalian, e.g. human serum, can additionally be determined. The active fragment or active analog can be bonded, or conjugated, to a moiety to improve its pharmacokinetics, for example one or more PEG molecules.

The active fragment of BRI2 peptide or active analog of a fragment of BRI2 peptide referred to herein can be administered by any means known in the art. The active fragment of BRI2 peptide or active analog of a fragment of BRI2 peptide referred to herein can be administered parentally, enterally or topically in a manner effective to enter the central nervous system of the subject. In an embodiment the active fragment of BRI2 peptide or active analog of a fragment of BRI2 peptide is administered directly into the central nervous system of the subject. In an embodiment the active fragment of BRI2 peptide or active analog of a fragment of BRI2 peptide is administered intranasally to the subject. In an embodiment the active fragment of BRI2 peptide or active analog of a fragment of BRI2 peptide is administered through the nasal upper epithelium of the subject. In an embodiment the active fragment of BRI2 peptide or active analog of a fragment of BRI2 peptide is administered through the olfactory epithelium. In embodiments, the active fragment of BRI2 peptide or active analog of a fragment of BRI2 peptide is administered into a cerebral ventricle of the subject or intrathecally to the subject. In an embodiment the active fragment of BRI2 peptide or active analog of a fragment of BRI2 peptide is administered via an implant. In an embodiment the implant is within the central nervous system of the subject. In an embodiment, the implant comprises a polymer matrix and the inhibitor is dispersed throughout the polymer matrix.

Caspases (cysteine-dependent aspartate-directed proteases) are a family of cysteine proteases that play essential roles in apoptosis. In relation to inhibition of a caspase in the present application, the relevant caspase is caspase-9 (the human form being Uniprot P55211 (CASP9_HUMAN)). In an embodiment, the inhibitor of caspase-9 is an inhibitor of human caspase-9. In an embodiment the inhibitor does not inhibit other caspases. In an embodiment the inhibitor does not inhibit human caspase-3. In an embodiment the inhibitor is selective for the caspase-9.

In an embodiment the inhibitor of caspase-9 is a small molecule. As used herein a “small molecule” refers to an organic compound characterized in that it contains several carbon-carbon bonds, and has a molecular weight of less than 2000 daltons. In an embodiment, the small molecule is less than 1500 daltons.

In an embodiment the inhibitor of caspase-9 is a peptide. In an embodiment the inhibitor is Z-LEHD-FMK (SEQ ID NO:11), wherein the “Z” and “FMK” are not amino acid residues but “Z” is carbobenzoxy- and the “FMK” is fluoromethylketone. The peptide (SEQ ID NO:11) can be O-methylated in the P1 position (D), or can be O-methylated in both the P1 position (D) and the P3 position (E). The peptides may be used, in a non-limiting embodiment, in the trifluoroacetic acid salt (TFA) salt form or a pharmaceutically acceptable salt form.

In an embodiment, the inhibitor is an inhibitor of caspase-9 and comprises XIAP-BIR3 domain (“XBIR3”). In an embodiment, the XBIR3 comprises SEQ ID NO:17. In an embodiment, the XBIR3 comprises SEQ ID NO:17 with 1, 2 or 3 additional amino acids at the N-terminus, C-terminus, or independently, at both termini.

In an embodiment, the inhibitor is an inhibitor of caspase-9 and is XIAP-BIR3 domain disulfide-linked to a cell-penetrating peptide. In an embodiment, the cell-penetrating peptide is Penetratin1 (“Pen1-XBIR3”). In an embodiment, the Penetratin1 comprises SEQ ID NO:18 (RQIKIWFQNRRMKWKK). In an embodiment the inhibitor of caspase-9 is SEQ ID NO:17 linked through a disulfide bond to SEQ ID NO:18. In an embodiment, SEQ ID NO:17 and SEQ ID NO:18 are in an equimolar ratio. In an embodiment the inhibitor of caspase-9 is SEQ ID NO:17 linked by a peptide bond to SEQ ID NO:18.

In an embodiment, the inhibitor of caspase-9 is RNAi-based. The inhibitor can be a shRNA or siRNA directed to a nucleic acid encoding a caspase-9. In an embodiment, the shRNA or siRNA is directed to o a nucleic acid encoding a human caspase-9, for example a nucleic acid encoding Uniprot P55211 (CASP9_HUMAN).

In an embodiment, the siRNA (small interfering RNA) as used in the methods or compositions described herein comprises a portion which is complementary to an mRNA sequence encoded by a gene encoding human caspase-9, and the siRNA is effective to inhibit expression of human caspase-9. In an embodiment, the siRNA comprises a double-stranded portion (duplex). In an embodiment, the siRNA is 20-25 nucleotides in length. In an embodiment the siRNA comprises a 19-21 core RNA duplex with a one or 2 nucleotide 3′ overhang on, independently, either one or both strands. The siRNA can be 5′ phosphorylated or not and may be modified with any of the known modifications in the art to improve efficacy and/or resistance to nuclease degradation. In an embodiment the siRNA can be administered such that it is transfected into one or more cells.

In one embodiment, a siRNA of the invention comprises a double-stranded RNA wherein one strand of the double-stranded RNA is 80, 85, 90, 95 or 100% complementary to a portion of an RNA transcript of a gene encoding human caspase-9. In another embodiment, a siRNA of the invention comprises a double-stranded RNA wherein one strand of the RNA comprises a portion having a sequence the same as a portion of 18-25 consecutive nucleotides of an RNA transcript of a gene encoding human caspase-9. In yet another embodiment, a siRNA of the invention comprises a double-stranded RNA wherein both strands of RNA are connected by a non-nucleotide linker. Alternately, a siRNA of the invention comprises a double-stranded RNA wherein both strands of RNA are connected by a nucleotide linker, such as a loop or stem loop structure.

In one embodiment, a single strand component of a siRNA of the invention is from 14 to 50 nucleotides in length. In another embodiment, a single strand component of a siRNA of the invention is 14, 15, 16, 17, 18, 19, 20, 21, 22, 23, 24, 25, 26, 27, or 28 nucleotides in length. In yet another embodiment, a single strand component of a siRNA of the invention is 21 nucleotides in length. In yet another embodiment, a single strand component of a siRNA of the invention is 22 nucleotides in length. In yet another embodiment, a single strand component of a siRNA of the invention is 23 nucleotides in length. In one embodiment, a siRNA of the invention is from 28 to 56 nucleotides in length. In another embodiment, a siRNA of the invention is 40, 41, 42, 43, 44, 45, 46, 47, 48, 49, 50, 51, or 52 nucleotides in length. In yet another embodiment, a siRNA of the invention is 46 nucleotides in length.

In another embodiment, an siRNA of the invention comprises at least one 2′-sugar modification. In another embodiment, an siRNA of the invention comprises at least one nucleic acid base modification. In another embodiment, an siRNA of the invention comprises at least one phosphate backbone modification.

In one embodiment, RNAi inhibition of human caspase-9 is effected by a short hairpin RNA (“shRNA”). The shRNA is introduced into the cell by transduction with a vector. In an embodiment, the vector is a lentiviral vector. In an embodiment, the vector comprises a promoter. In an embodiment, the promoter is a U6 or H1 promoter. In an embodiment the shRNA encoded by the vector is a first nucleotide sequence ranging from 19-29 nucleotides complementary to the target gene, in the present case human caspase-9. In an embodiment the shRNA encoded by the vector also comprises a short spacer of 4-15 nucleotides (a loop, which does not hybridize) and a 19-29 nucleotide sequence that is a reverse complement of the first nucleotide sequence. In an embodiment the siRNA resulting from intracellular processing of the shRNA has overhangs of 1 or 2 nucleotides. In an embodiment the siRNA resulting from intracellular processing of the shRNA overhangs has two 3′ overhangs. In an embodiment the overhangs are UU.

In an embodiment the siRNA or shRNA is targeted to the central nervous system of the subject.

In an embodiment the inhibitor of caspase-9 is an antibody or a fragment of an antibody, which antibody or fragment of an antibody is able to access a cell of the central nervous system and act intracellularly. As used herein, the term “antibody” refers to complete, intact antibodies. As used herein “antibody fragment” refers to Fab, Fab′, F(ab)2, and other antibody fragments, which fragments (like the complete, intact antibodies) bind the antigen of interest, in this case an inhibitor of apoptosis protein. Complete, intact antibodies include, but are not limited to, monoclonal antibodies such as murine monoclonal antibodies, polyclonal antibodies, chimeric antibodies, human antibodies, and humanized antibodies.

Various forms of antibodies may be produced using standard recombinant DNA techniques (Winter and Milstein, Nature 349: 293-99, 1991). For example, “chimeric” antibodies may be constructed, in which the antigen binding domain from an animal antibody is linked to a human constant domain (an antibody derived initially from a nonhuman mammal in which recombinant DNA technology has been used to replace all or part of the hinge and constant regions of the heavy chain and/or the constant region of the light chain, with corresponding regions from a human immunoglobulin light chain or heavy chain) (see, e.g., Cabilly et al., U.S. Pat. No. 4,816,567; Morrison et al., Proc. Natl. Acad. Sci. 81: 6851-55, 1984). Chimeric antibodies reduce the immunogenic responses elicited by animal antibodies when used in human clinical treatments. In addition, recombinant “humanized” antibodies may be synthesized. Humanized antibodies are antibodies initially derived from a nonhuman mammal in which recombinant DNA technology has been used to substitute some or all of the amino acids not required for antigen binding with amino acids from corresponding regions of a human immunoglobulin light or heavy chain. That is, they are chimeras comprising mostly human immunoglobulin sequences into which the regions responsible for specific antigen-binding have been inserted (see, e.g., PCT patent application WO 94/04679). Animals are immunized with the desired antigen, the corresponding antibodies are isolated and the portion of the variable region sequences responsible for specific antigen binding are removed. The animal-derived antigen binding regions are then cloned into the appropriate position of the human antibody genes in which the antigen binding regions have been deleted. Humanized antibodies minimize the use of heterologous (inter-species) sequences in antibodies for use in human therapies, and are less likely to elicit unwanted immune responses. Primatized antibodies can be produced similarly.

Another embodiment of the antibodies and fragments of antibodies employed in the compositions and methods of the invention is a human antibody, which can be produced in nonhuman animals, such as transgenic animals harboring one or more human immunoglobulin transgenes. Such animals may be used as a source for splenocytes for producing hybridomas, as is described in U.S. Pat. No. 5,569,825.

The term “human antibody”, as used herein, is intended to include antibodies having variable regions in which both the framework and CDR regions are derived from sequences of human origin. Furthermore, if the antibody contains a constant region, the constant region also is derived from such human sequences, e.g., human germline sequences, or mutated versions of human germline sequences. The human antibodies of the invention may include amino acid residues not encoded by human sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The term “recombinant human antibody”, as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the human antibody, e.g., from a transfectoma, antibodies isolated from a recombinant, combinatorial human antibody library, and antibodies prepared, expressed, created or isolated by any other means that involve splicing of all or a portion of a human immunoglobulin gene, sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

Antibody fragments and univalent antibodies may also be used in the methods and compositions of this invention. Univalent antibodies comprise a heavy chain/light chain dimer bound to the Fc (or stem) region of a second heavy chain. “Fab region” refers to those portions of the chains which are roughly equivalent, or analogous, to the sequences which comprise the Y branch portions of the heavy chain and to the light chain in its entirety, and which collectively (in aggregates) have been shown to exhibit antibody activity. A Fab protein includes aggregates of one heavy and one light chain (commonly known as Fab′), as well as tetramers which correspond to the two branch segments of the antibody Y, (commonly known as F(ab)₂), whether any of the above are covalently or non-covalently aggregated, so long as the aggregation is capable of specifically reacting with a particular antigen or antigen family.

The antibody, or fragment, can be of e.g., any of an IgA, IgD, IgE, IgG, or IgM antibody. In an embodiment the antibody is an immunoglobulin G. In an embodiment the antibody fragment is a fragment of an immunoglobulin G. In an embodiment the antibody is an IgG1, IgG2, IgG2a, IgG2b, IgG3 or IgG4. The IgA antibody can be, e.g., an IgA1 or an IgA2 antibody. A combination of any of these antibodies subtypes can also be used. One consideration in selecting the type of antibody to be used is the desired serum half-life of the antibody. IgG has a serum half-life of 23 days, IgA 6 days, IgM 5 days, IgD 3 days, and IgE 2 days. (Abbas A K, Lichtman A H, Pober J S. Cellular and Molecular Immunology, 4th edition, W.B. Saunders Co., Philadelphia, 2000). Another consideration is the size of the antibody or fragment. For example, the size of IgG is smaller than that of IgM allowing for greater penetration into certain tissues.

The inhibitor of caspase-9 referred to herein can be administered by any means known in the art. The inhibitor of caspase-9 referred to herein can be administered parentally, enterally or topically in a manner effective to enter the central nervous system of the subject. In an embodiment the inhibitor of caspase-9 is administered directly into the central nervous system of the subject. In an embodiment the inhibitor of caspase-9 is administered intranasally to the subject. In an embodiment the inhibitor of caspase-9 is administered through the nasal upper epithelium of the subject. In an embodiment the inhibitor of caspase-9 is administered through the olfactory epithelium. In embodiments, the inhibitor of caspase-9 is administered into a cerebral ventricle of the subject or intrathecally to the subject. In an embodiment the inhibitor of caspase 9 is administered via an implant. In an embodiment the implant is within the central nervous system of the subject. In an embodiment, the implant comprises a polymer matrix and the inhibitor is dispersed throughout the polymer matrix.

The inhibitors, active fragments, active analogs of fragments, and agents described herein can be administered to the subject in a pharmaceutical composition comprising a pharmaceutically acceptable carrier. The pharmaceutically acceptable carrier used can depend on the route of administration. As used herein, a “pharmaceutically acceptable carrier” is a pharmaceutically acceptable solvent, a suspending vehicle, for delivering the instant agents to the animal or human subject. The carrier may be liquid or solid and is selected with the planned manner of administration in mind Liposomes are also a pharmaceutically acceptable carrier. Pharmaceutically acceptable carriers are known in the art, and include, but are not limited to, additive solution-3 (AS-3), saline, phosphate buffered saline, Ringer's solution, lactated Ringer's solution, Locke-Ringer's solution, Krebs Ringer's solution, Hartmann's balanced saline solution, and heparinized sodium citrate acid dextrose solution. In an embodiment the pharmaceutical carrier is acceptable for administration into the central nervous system of a mammal.

The inhibitors, active fragments, active analogs of fragments, and agents can be administered together or independently in admixtures with suitable pharmaceutical diluents, extenders, excipients, or carriers (collectively referred to herein as a pharmaceutically acceptable carrier) suitably selected with respect to the intended form of administration and as consistent with conventional pharmaceutical practices.

Techniques and compositions for making dosage forms useful in the invention are described-in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.). All of the aforementioned publications are incorporated by reference herein.

Dosing can be any method or regime known in the art. For example, twice daily, daily, weekly, monthly, as needed, and continuously.

Production of sAPPβ from APP by a β-secretase can be quantified by any technique known in the art, for example by measuring sAPPβ produced by β-secretase in media by Western blot. Anti-sAPPβ antibodies are commercially available and can be employed.

In a non-limiting embodiment, binding of an agent to β-CTF can be determined by antibody-based detection techniques.

In a non-limiting embodiment, inhibition of a caspase, for example caspase-9 or caspase-3, can be measured by any technique known in the art, including fluorimetric or colorimetric detection of cleavage of caspase-specific substrates. Alternatively, detectable agents that bind only to activated caspases can be used. For example, a synthetic substrate specific for Caspase-9 is FITC-LEHD-FMK which binds to activated caspase-9 in apoptotic cells and can be detected by fluorescence microscopy or flow cytometry (excitation 485 nm, emission 535 nm). Immunosorbent enzyme assay fluorometric-based techniques may also be used, with detectable specific antibodies, e.g. anti-caspase 3-specific monoclonal capture antibody or anti-caspase 9-specific monoclonal capture antibody in combination with a specific caspase-3 or caspase-9 substrate, respectively.

The invention also provides a method of identifying a molecule that inhibits APP processing comprising a) modeling in silico (i) the 3-dimensional site or sites on SEQ ID NO:4, 5, 9, 10 or 12 that bind APP, or (ii) the 3-dimensional site or sites on APP which bind SEQ ID NO:4, 5, 9, 10 or 12; b) testing in silico if a compound from a library of compounds (i) binds to the modeled 3-dimensional site on APP or (ii) mimics the 3-dimensional site or sites on SEQ ID NO:4, 5, 9, 10 or 12 that bind APP, and c) determining in vitro if a chemically stable small molecule identified as (i) binding to the site or sites in silico or (ii) mimicking the 3-dimensional site or sites on SEQ ID NO:4, 5, 9, 10 or 12 in b), binds to APP and inhibits its processing. In silico modeling of 3-D binding sites for rational drug design is known in the art. For example, see Computational Resources for Protein Modelling and Drug Discovery Applications, Infectious Disorders—Drug Targets (2009), 9, 557-562, B. Dhaliwal and Y. W. Chen, the contents of which are hereby incorporated by reference. Mapping the binding site of N3-2A on APP permits identification of the residues in N3-2A that are important for binding. An NMR-based fragment screen can identify small molecules that bind to the N3 site. Structural NMR studies can position small molecule portions on APP so that they can be linked together to form more potent binders. Efficacy in reversing the behavioral/memory impairments can readily be determined in the Tg2576 mouse model of AD, and in the recently developed FDDKI mice.

Also provided is a method of identifying a small molecule that inhibits APP processing comprising determining in vitro if a small molecule identified as (i) binding to the site or sites of APP previously determined to be bound by SEQ ID NO:4, 5, 9, 10 or 12, or (ii) mimicking the 3-dimensional site or sites on SEQ ID NO:4, 5, 9, 10 or 12 that bind APP, binds to APP and inhibits its processing.

The methods disclosed herein can be used with any mammalian subject. Preferably, the mammal is a human.

All combinations of the various elements described herein are within the scope of the invention unless otherwise indicated herein or otherwise clearly contradicted by context.

This invention will be better understood from the Experimental Details, which follow. However, one skilled in the art will readily appreciate that the specific methods and results discussed are merely illustrative of the invention as described more fully in the claims that follow thereafter.

### Example I

### Caspase-9 Inhibition for Treating Dementia

### Introduction

Mouse models of human dementia invariably use transgenic expression systems that do not reflect the genotypes of human disease and cannot replicate loss of function amyloidosis (Jucker, 2010; Morrissette et al, 2009). Therefore, a knock-in (KI) mouse model of FDD (FDDKI) was generated that is genetically congruous with the human disease. FDD is caused by a 10-nucleotide duplication preceding the stop codon of the BRI2/ITM2B gene (Vidal et al, 2000). In normal individuals, BRI2 is synthesized as an immature type-II membrane protein (imBRI2) that is cleaved at the C-terminus by a pro-protein convertase to produce mature BRI2 (mBRI2) and a 23-aa soluble C-terminal fragment (CTF) (Bri23) (Choi et al, 2004). However, in FDD patients, a longer CTF, the ADan peptide (Vidal et al, 2000) is generated from the Danish mutant protein (BRI2-ADan), which has amyloidogenic properties. ADan forms amyloid angiopathy in the small blood vessels and capillaries of the cerebrum, choroid plexus, cerebellum, spinal cord and retina (Vidal et al, 2000). FDD patients also show diffuse brain atrophy, particularly in the cerebellum, cerebral cortex and white matter, as well as the presence of very thin and almost demyelinated cranial nerves; neurofibrillary tangles are the major histological finding in the hippocampus (Vidal et al, 2000).

FDDKI mice present reduced BRI2 levels, impaired synaptic plasticity and severe hippocampal memory deficits. These animals show no cerebral lesions that are reputed characteristics of human dementia, such as tangles or amyloid plaques. Bri2+/− mice exhibit synaptic and memory deficits similar to FDDKI mice, and memory loss of FDDKI mice is prevented by expression of WT BRI2, indicating that Danish dementia is caused by loss of BRI2 function. These results indicated that the Danish BRI2 mutation underlies abnormal memory due to loss of BRI2 function and independently of histopathological alterations typically evident in advanced neurodegenerative disease. Remarkably, APP haplodeficiency prevents memory and synaptic dysfunctions, consistent with a role for APP metabolites in the pathogenesis of memory and synaptic deficits. This genetic suppression provides compelling evidence that APP and BRI2 functionally interact, and that the neurological effects of the Danish form of BRI2 only occur when sufficient levels of APP are supplied by two alleles. Moreover, recent studies in the laboratory further stress the importance of APP and APP processing in FDD. Importantly, APP processing is genetically linked to AD pathogenesis.

Currently, therapies for AD are being tested on transgenic mice carrying mutant APP, PSEN1/2 or BRI2/ITM2b, since amyloidogenic peptides are considered the pathogenic factor in dementias (Hardy & Selkoe, 2002), and over-expression is necessary to reproduce amyloidosis (Jucker, 2010; Morrissette et al, 2009). However, over-expression of mutant genes might produce harmful effects unrelated to dementias, leading to erroneous information concerning pathogenesis and therapy of human diseases. The clinical failures of compounds efficacious in transgenic models support this hypothesis (Ganjei, 2010). To avoid artifacts of over-expression, the above-described knock-in mouse model of FDD was used in the present studies. Moreover, many APP-derived fragments have been linked to activation of caspases and apoptotic pathways. These include Aβ, AID (Passer et al, 2000), C31 (Lu et al, 2000), Jcasp (Bertrand et al, 2001; Madeira et al, 2005) and a fragment derived from sAPPβ (Nikolaev et al, 2009). Given the increase in synaptic APP fragments (such as AID and sAPPβ) observed in FDDKI mice (Tamayev et al, 2011), caspase activation was tested.

### Materials.

Pan Caspase fmk Inhibitor Z-VAD (Z-V-A-D(OMe)-FMK), Caspase-3 fmk Inhibitor Z-DEVD (Z-D(OMe)-E(OMe)-V-D(OMe)-FMK (SEQ ID NO:14), Caspase-9 fmk Inhibitor Z-LEHD (Z-L-E(OMe)-H-D(OMe)-FMK.TFA) (SEQ ID NO:13) were obtained from R&D systems (Minneapolis, Minn.) as Cat. Nos. FMK001, FMK004 and FMK008, respectively.

### Experimental Results

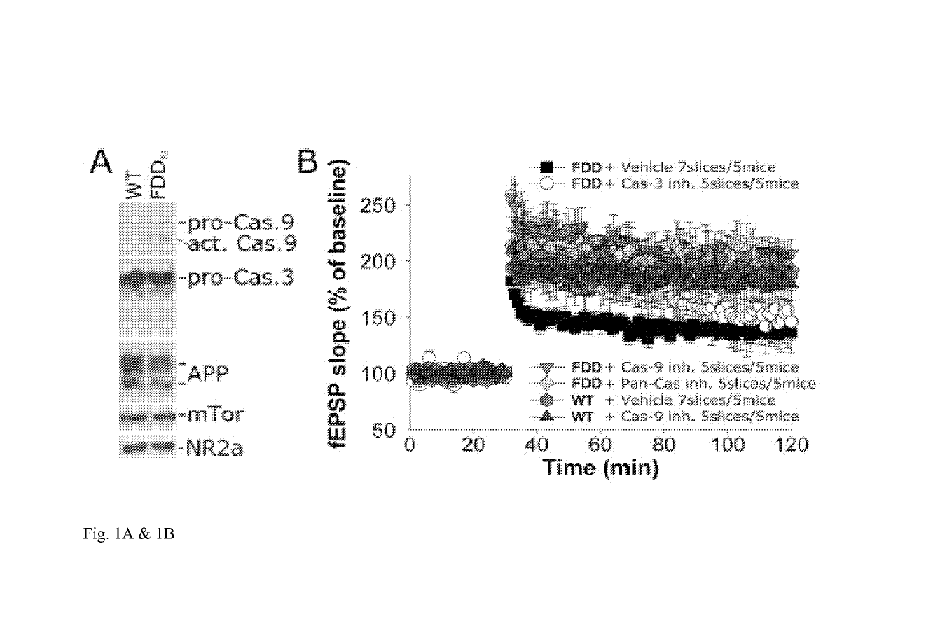

As shown in FIG. 1A, the basal levels of active caspase-9 are elevated in FDDKI mice as compared to WT littermates. Activation of caspase-3, caspase-7 and caspase-6 (not shown) could however not be detected. An inhibitor of caspase-9 (but not an inhibitor of caspase-3) corrected the LTP deficits of FDDKI mice, supporting a role for caspase-9 in the genesis of the synaptic defect in FDDKI mice (FIG. 1B). It is important to notice that FDDKI mice do not present obvious neuronal loss. This is consistent with the finding that the executioner caspases-3 and -7, which mediate apoptosis, are not active and suggests that activation of the apical caspase-9 is below the threshold for activation of cell death pathways. Alternatively, caspase-9 activation occurs in cellular sub-domains that are deprived or scarcely supplied with executioner caspases or rich in inhibitors of caspase-3/7. It should also be noted that a role for caspase-3 in Long Term Depression (LTD) has recently been described (Li et al, 2010), stressing the concept that executioner caspases do not necessarily lead to cell death (Galluzzi et al, 2008).

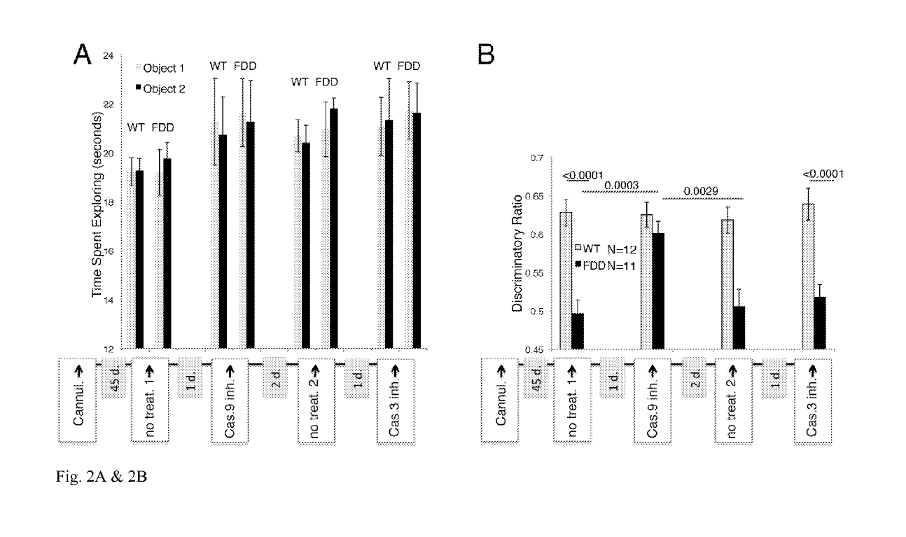

To test whether caspase-9 has a role in the pathogenesis of memory deficits, a pharmacological approach was employed. Intra-cerebral ventricular (ICV) administration of the caspase-9 inhibitor Z-LEHD-FMK (SEQ ID NO:13) and, as controls, of the Caspase-3 inhibitor ZDEVD-FMK or the vehicle alone, were performed. 12 WT and 11 FDD mice were used for each compound when the mice were 9 months of age, which is the age at which FDDKI mice present full memory impairments. The mice were injected with 1 μl of a 500 μM solution in PBS of each caspase inhibitor or vehicle 1 hr before each novel object recognition (NOR) test was performed. NOR is a non-aversive task that relies on the mouse's natural exploratory behavior. The test is performed, over two days, by placing each mouse into a 40 cm×40 cm open field chamber with 2 feet high opaque walls with 2 identical objects, on day 1, spaced equally from each other and the walls of the chamber. The mice are given sufficient time to explore both objects, and on day 2, one of the objects is replaced into a different shaped object, now called the novel object. The mouse's natural explorative behavior should have the mouse spend more time exploring the new object, rather than the old one. It was found that the FDDKI mice still spent the same amount of time exploring the two objects as if they were both novel to them, while the WT mice spent more time exploring the novel object compared with the object used 24 h prior, as expected (FIG. 2A).

Results were recorded as an object discrimination ratio (ODR), which is calculated by dividing the time the mice spent exploring the novel object by the total amount of time exploring the two objects. This finding confirms that memory is impaired in FDDKI mice in an ethologically relevant, non-aversive behavioral context. Subsequently, mice were injected in the lateral ventricle with caspase-9 inhibitor and tested again. Treated FDDKI mice spent significantly more time exploring the novel object just as caspase-9 inhibitor treated controls (FIG. 2b). A further NOR test showed that, without treatment, FDDKI mice relapsed into amnesia (FIG. 2b), demonstrating that the therapeutic effect of caspase-9 inhibitor is reversible and short-lived.

Next, the behavioral effects of caspase-3 inhibition were analyzed. The caspase-3 inhibitor neither improved memory of FDDKI mice nor altered performance of WT animals (FIG. 2b). Thus, it is concluded that pathological action of caspases-9 impairs the normal formation of memory. Recent reports indicate that caspase-3, but not caspase-9, is required for LTP impairments caused by APP over-expression and Aβ42 (D'Amelio et al, 2010; Jo et al, 2011). The present data suggests that caspase-3 is activated in mouse models characterized by the indiscriminate over-expression of human mutant APP and Aβ42. It is probable that these phenomena represent artificial effects unrelated to human dementia.

Accordingly, a pathological activation of Caspase-9 during memory formation is an essential factor in causing memory loss and dementia and countering that activation provides therapies for treating memory loss and dementia.

### Example II

### Selective Caspase 9 Inhibition

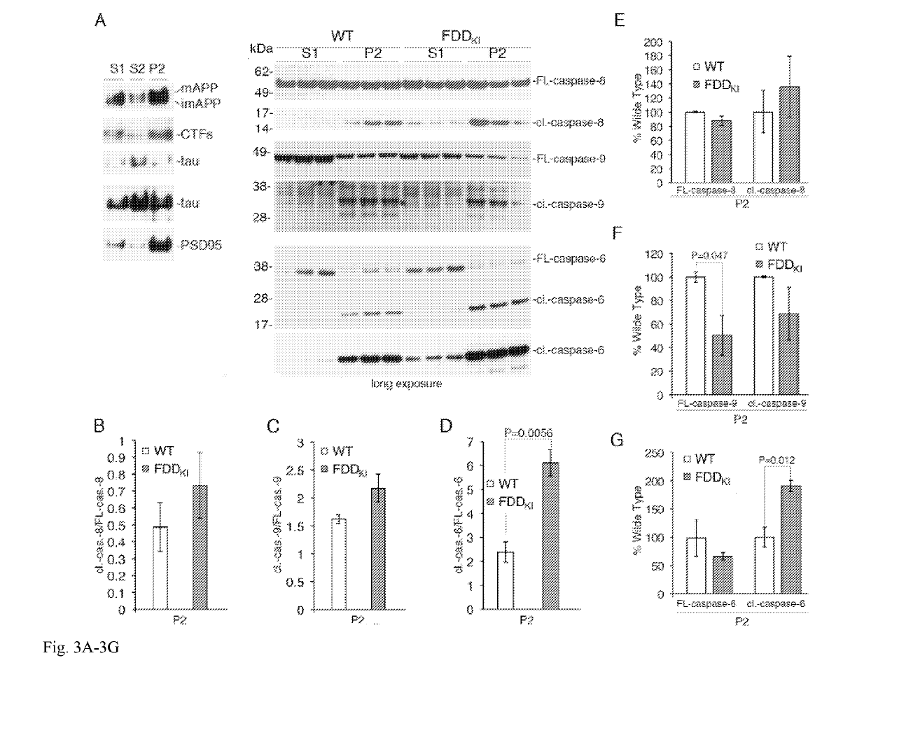

The initiator caspase-9 is hyperactive in FDDKI mice hippocampal synaptic fractions. Based on the evidence that caspases are pathogenic in FDDKI mice, biochemical evidence was sought of caspase activation and/or activity. Because FDDKI mice have deficits in hippocampal-dependent memory and synaptic activity, which are associated with learning and memory, it was tested whether signs of caspase activation were detectable in hippocampal synaptic preparations of 12 month-old mice. As shown in FIG. 3A, the P2 fraction, which represents the crude synaptosomal fraction, was enriched in PSD95, a synaptic protein, while the S2 fraction, containing cytosol, soluble proteins and light membrane, was enriched in tau.

Caspases are synthesized as zymogens (FL-caspase). Effector caspases are cleavage by initiator caspases (cl.-caspase) and this cleavage leads to activation of effector caspases. Presence of cl.-effector caspases is thereby indicative of caspase activation in a preparation (McStay et al, 2008). It was observed that hippocampal synaptosomal fractions (P2) of both WT and FDDKI mice are highly enriched in cl.-caspase-6 fragments (FIG. 3B) as compared to total homogenates (S1) indicating that caspases are normally active in hippocampal synaptic compartments of 12-month-old mice. The levels of cl.-caspase-6 are significantly higher in synaptic preparations of FDDKI mice as compared to WT littermates (FIGS. 3B and C), while cl.-caspase-3 was detectable in hippocampal synaptic preparations from FDDKI mice but not WT animals (FIG. 3B). Moreover, cl.-caspase-3 and cl.-caspase-6 were also detected in S1 fractions of FDDKI but not WT mice (FIG. 3B). These data show that caspase activation is increased in Danish mice hippocampal synaptosomes.

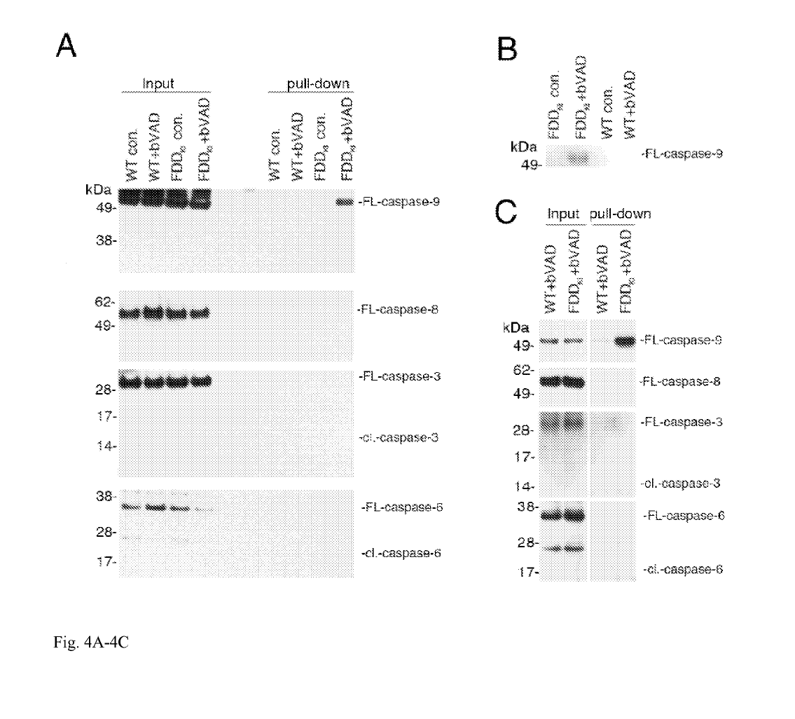

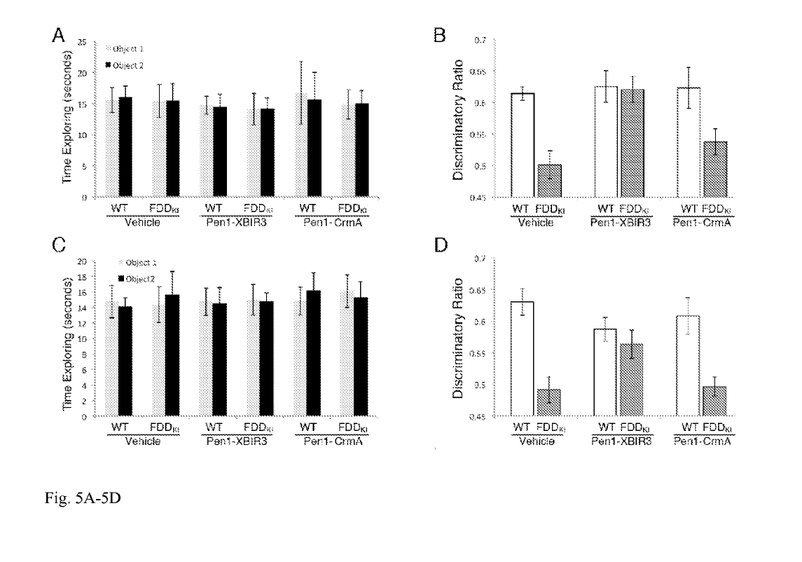

The evidence that levels of cleaved effector caspases are higher in hippocampal synaptic fractions of FDDKI mice than in WT mice suggests that the activity of initiator caspases is increased in the hippocampal synaptic compartments of FDDKI mice. As noted above initiator caspase are activated by dimerization and the analysis of cleaved caspase fragments does not measure the activity of initiator caspases. To allow unequivocal identification of active caspase an unbiased in vivo active caspase-trapping assay was used (Akpan et al, 2011). The caspase activity probe bVAD is the best way to determine whether caspases are active since bVAD binds irreversibly to all caspases that are active. In other words, if a caspase is active and its active site is available, bVAD will bind to it. Because bVAD is biotinylated, it can be isolated on streptavidin agarose along with any active caspase that is bound to it. This strategy has also the advantage of enriching for the apical active caspase rather than the downstream caspases in a pathway that involves a cascade of caspase activation. To determine which caspases are active, FDDKI and WT mice were injected in one hippocampus with 100 nmol of bVAD. In these experiments, 6 (FIG. 4A) or 5 (FIG. 4B) month-old mice were utilized since the memory deficits of FDDKI mice start at around 4-5 months of age (Tamayev et al, 2010b). Two hrs post treatment, the injected region and the contralateral non-injected area were dissected, and bVADcaspase complexes were isolated on streptavidin-agarose beads and analyzed by Western blotting. bVAD captured greatly more FL-caspase-9, but not FL-caspase-8, from the hippocampus of the FDDKI sample as compared to the WT littermate sample (FIG. 4A). The binding was specific because streptavidin-agarose beads did not pull-down active FL-caspase-9 from homogenates prepared from the contralateral, non-injected sample. Cl.-caspase-3 and -6 were not trapped by bVAD (FIG. 4A). The inability to isolate cl.-caspase-3 and cl.-caspase-6 may depend on the fact that bVAD inhibits caspase-9 activity, thereby inhibiting processing of effector caspases-3 and -6 by active caspase-9. This possibility is not very likely because in FDDKI mice there is probably ongoing caspase activation and bVAD will bind to any active caspase present at the moment of bVAD administration. Alternatively, cl.-caspase-3 may not be available for bVAD-binding because it is complexed in vivo with endogenous inhibitor of apoptosis proteins (IAPs). Lastly, cl.-caspase-3 and cl.-caspase-6 may be captured by bVAD at very low levels that are below the detection power of our experimental system. This is indeed a possibility given the low level of material that can be harvested in this experimental setting and the evidence that cl-caspase-3 and cl-caspase-6 are not detectable in the input material either.

To determine whether active caspase-9 was present in synaptic fractions, the experiment was repeated and performed bVAD pull-downs from synaptosomal fractions. As shown in FIG. 4B, active caspase-9 was also isolated from synaptosomal fractions of FDDKI but not WT mice. Blotting for caspase-3, -6 and -8 showed once more absence of detectable active caspase-3, -6 or -8 in these synaptosomal preparations (data not shown).

To formally exclude that the differences between WT and FDDKI mice illustrated above did not depend on disparity of bVAD delivery in vivo, organotypic hippocampal cultures were prepared from 5 month-old WT and FDDKI mice. Once again, bVAD trapped significantly more active caspase-9 from organotypic hippocampal culture of FDDKI mice than WT littermates (FIG. 4C). Once again, active FL-caspase-8, cl.-caspase-3 and cl.-caspase-6 neither in WT nor in FDDKI sample could not be detected. Altogether these data indicate that caspase-9 is excessively activated in Danish dementia mice. Moreover, the data suggest that, if the Danish mutation triggers a cascade of caspase activation, caspase-9 is the apical caspase in such a cascade.

Specific inhibition of caspase-9 with Pen1-XBIR3 provides therapeutic rescue of the object recognition deficit. The findings that reducing caspase activity with commercial peptide inhibitors rescues synaptic/memory deficits and that caspase-9 is active in FDDKI mice, suggest that caspase-9 is involved in the pathogenesis of these deficits. To specifically determine the functional relevance of caspase-9 activity in memory loss pathogenesis, caspase-9 was specifically inhibited. As a control, a specific inhibitor of caspase-8 was also used activity. Mammals express a family of cell death inhibiting proteins known as IAPs. IAPs contain BIR domains, which perform specific functions. One member of this family, XIAP, is a potent specific inhibitor of active caspase-9, caspase-3, and caspase-7. The XIAP-BIR3 domain is a specific inhibitor of active caspase-9, and the XIAP-BIR2-linker domain inhibits active caspase-3 and caspase-7 (Eckelman et al, 2006). Serpins are also caspases inhibitors and CrmA (a cowpox serpin) inhibits caspase-8 (as well as caspase-1, which is involved in inflammatory responses) but not other murine caspases (Garcia-Calvo et al, 1998). To provide intracellular delivery, XIAP-BIR3 and CrmA were disulfide-linked to Penetratin1 (Pen1), a cell-penetrating peptide (Akpan et al, 2011). Upon entry into the cell the reducing environment of the cytoplasm reduces the disulfide linkage. This releases the peptide cargo and allows it to act at its target. Pen1-XBIR3 also inhibits caspase-9 dependent cell death in primary hippocampal neuron cultures, and Pen1-XBIR3 delivery to the CNS blocks caspase-9 in an in vivo model of cerebral ischemia (Akpan et al, 2011).

NOR experiments were used to assess the effect of Pen1-XBIR3 on memory. Six groups of mice (3 groups of FDDKI mice and 3 groups of WT littermates) were injected in the lateral ventricle either with vehicle alone, Pen1-XBIR3 or Pen1-CrmA 1 hr before the training/testing trials. Pen1-XBIR3 treated FDDKI mice spent significantly more time exploring the novel object showing reversal of the memory deficits (FIGS. 5A and B). On the contrary, Pen1-CrmA treated FDDKI mice showed memory deficits comparable to those observed in vehicle-treated FDDKI mice. Neither Pen1-XBIR3 nor Pen1-CrmA altered memory in WT animals. Following 5 days of rest, a new NOR test performed without treatments showed that the therapeutic effect of Pen1-XBIR3 persisted for at least 5 days post injection (FIGS. 5C and D). This lab's previous studies showed that one dose of Pen1-XBIR3 provided functional protection against ischemia for 3 weeks post-infarction (Akpan et al, 2011). Thus, Pen1-XBIR3 rescued the memory deficit of FDDKI mice, while Pen1-CrmA did not. These data indicate that excessive activation of caspase-9 in FDDKI mice is an essential step in the pathogenesis of memory loss.

Methods: Pen1 (Q-Biogene; PENB0500 Biotinylated Activated Penetratin 1 Peptide) was mixed at an equimolar ratio with purified XBIR3 and incubated overnight at 37° C. to generate disulfide-linked Pen1-XBIR3. Linkage was assessed by 20% SDS-PAGE and Western blotting with anti-His antibody.

Sequence of BIR3
(SEQ ID NO: 15)
SDAVSSDRNF PNSTNLPRNP SMADYEARIF TFGTWIYSVN
KEQLARAGFY ALGEGDKVKC FHCGGGLTDW KPSEDPWEQH
AKWYPGCKYL LEQKGQEYIN NIHLTHSLEE CLVRTT
Sequence of XIAP(SEQ ID NO: 16):

| | |
|---|---|
| 1 | MTFNSFEGSK TCVPADINKE EEFVEEFNRL |
| | KTFANFPSGS PVSASTLARA GFLYTGEGDT |
| 61 | VRCFSCHAAV DRWQYGDSAV GRHRKVSPNC |
| | RFINGFYLEN SATQSTNSGI QNGQYKVENY |
| 121 | LGSRDHFALD RPSETHADYL LRTGQVVDIS |
| | DTIYPRNPAM YSEEARLKSF QNWPDYAHLT |
| 181 | PRELASAGLY YTGIGDQVQC FCCGGKLKNW |
| | EPCDRAWSEH RRHFPNCFFV LGRNLNIRSE |
| 241 | SDAVSSDRNF PNSTNLPRNP SMADYEARIF |
| | TFGTWIYSVN KEQLARAGFY ALGEGDKVKC |
| 301 | FHCGGGLTDW KPSEDPWEQH AKWYPGCKYL |
| | LEQKGQEYIN NIHLTHSLEE CLVRTTEKTP |
| 361 | SLTRRIDDTI FQNPMVQEAI RMGFSFKDIK |
| | KIMEEKIQIS GSNYKSLEVL VADLVNAQKD |
| 421 | SMQDESSQTS LQKEISTEEQ LRRLQEEKLC |
| | KICMDRNIAI VFVPCGHLVT CKQCAEAVDK |
| 481 | CPMCYTVITF KQKIFMS |

XIAP
(SEQ ID NO: 16)
showing BIR3 domain (aa 241-356) underlined.
(SEQ ID NO: 17)

| | |
|---|---|
| 1 | MTFNSFEGSK TCVPADINKE EEFVEEFNRL |
| | KTFANFPSGS PVSASTLARA GFLYTGEGDT |
| 61 | VRCFSCHAAV DRWQYGDSAV GRHRKVSPNC |
| | RFINGFYLEN SATQSTNSGI QNGQYKVENY |
| 121 | LGSRDHFALD RPSETHADYL LRTGQVVDIS |
| | DTIYPRNPAM YSEEARLKSF QNWPDYAHLT |
| 181 | PRELASAGLY YTGIGDQVQC FCCGGKLKNW |
| | EPCDRAWSEH RRHFPNCFFV LGRNLNIRSE |
| 241 | SDAVSSDRNF PNSTNLPRNP SMADYEARIF |
| | TFGTWIYSVN KEQLARAGFY ALGEDKVKC |
| 301 | FHCGGGLTDW KPSEDPWEQH AKWYPGCKYL |
| | LEQKGQEYIN NIHLTHSLEE CLVRTTEKTP |
| 361 | SLTRRIDDTI FQNPMVQEAI RMGFSFKDIK |
| | KIMEEKIQIS GSNYKSLEVL VADLVNAQKD |
| 421 | SMQDESSQTS LQKEISTEEQ LRRLQEEKLC |
| | KICMDRNIAI VFVPCGHLVT CKQCAEAVDK |
| 481 | CPMCYTVITF KQKIFMS |

### Discussion

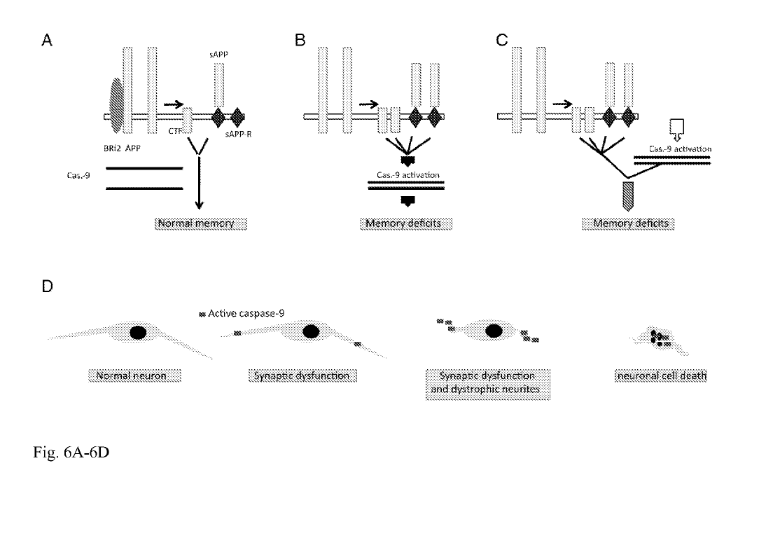

If activation of caspase-9 is confined to synaptic compartments, as it is the case for FDDKI mice, aberrant caspase-9 activation may lead to synaptic-memory deficits and dystrophy of neurites but not to neuronal cell death, explaining why FDDKI mice do not present overt neurodegeneration in spite of high caspase-9 activity (FIG. 6D). However, if activation of caspase-9 is recurring and sustained, as may be the case for dementia patients, the probability that eventually, in any given neuron, active caspase-9 may leak into the neuronal cell body triggering effector caspases and leading to genomic DNA fragmentation will be greater in patients rather that normal individuals (FIG. 6D). Over time, these changes can result in neuronal loss and neuritic dystrophy that are typical features of advanced neurodegenerative diseases.

This study is consistent with inhibiting caspase-9 activity as a viable therapeutic option in human dementias. Here, intraventricular administration of Pen1-XBIR3 was used that provides direct delivery to the brain. In a previous paper, this laboratory has shown that direct parenchymal or intranasal delivery of Pen1-XBIR3 is therapeutically effective in rat models of stroke (Akpan et al, 2011). From a therapeutic perspective, intranasal delivery is a very attractive treatment strategy for CNS disorders because it provides direct, noninvasive access to the brain via the olfactory pathway.

### Example III

### BRI2 Peptides for Treating Dementia

### Introduction

Amyloid deposition of Aβ peptide characterizes AD. Aβ derives from sequential cleavage of APP by β- and γ-secretases (Cole & Vassar, 2007; De Strooper et al, 2010). Interestingly, mutations in either APP or the γ-secretase genes PSEN1/2 cause familial AD (FAD) (Bertram et al, 2010; St George-Hyslop & Petit, 2005). Mutation of BRI2/ITM2b causes FDD, an AD-like familial dementia with amyloid deposits. The FDD plaques contain Aβ and ADan, which derives from processing of mutant BRI2 by convertases (Vidal et al, 2000; Choi et al, 2004). Since amyloidogenic peptides are believed to cause dementias (Hardy & Selkoe, 2002), transgenic mice carrying mutant APP, PSEN1/2 or BRI2/ITM2b are used to model these dementias, as over-expression is necessary to reproduce amyloidosis (Jucker, 2010). However, over-expression of mutant genes might produce harmful effects unrelated to dementias and lead to erroneous information concerning pathogenesis and therapy of human diseases. The clinical failures of compounds efficacious in transgenic models support this hypothesis (Ganjei, 2010). To avoid artifacts of over-expression, a knock-in mouse model of FDD (FDDKI) was generated that, like FDD patients (Vidal et al, 2000), is heterozygous for one mutated FDD allele of BRI2/ITM2b (Giliberto et al, 2009). FDDKI mice develop progressive synaptic and memory deficits due to loss of Bri2, with no amyloidosiys (Tamayev et al, 2010b).

BRI2 binds APP and inhibits APP processing (Fotinopoulou et al, 2005; Matsuda et al, 2005; Matsuda et al, 2008; Matsuda et al, 2011a); owing to the loss of BRI2, APP processing is increased in FDD (Matsuda et al, 2011b; Tamayev et al, 2011). Remarkably, memory and synaptic deficits of FDDKI mice require APP (Tamayev et al, 2011), providing genetic evidence that APP and BRI2 functionally interact, and that APP mediates FDD neuropathology.

### Material And Methods

Cells, plasmids and reagents. Cells, transfection methods, APP expression construct and luciferase assays were described (Matsuda et al, 2005; Scheinfeld et al, 2002). BRI2 fragments were PCR-amplified and cloned into pcDNA3mycHisB (Invitrogen). The following antibodies were used: α-APP (22C11/Chemicon); α-sAPPα and α-sAPPβ (IBL); α-APPCTF (Invitrogen/Zymed); α-myc (Cell-Signaling); anti-α-Tubulin (Sigma); Flag-M2-agarose-beads (Sigma); secondary antibodies (Southern Biotechnology); β-secretase-Inhibitor IV and Compound-e (Calbiochem); streptavidin-agarose-beads (Sigma). β-secretase activity was tested using the Invitrogen FRET assay kit following the manufacturer's instructions.

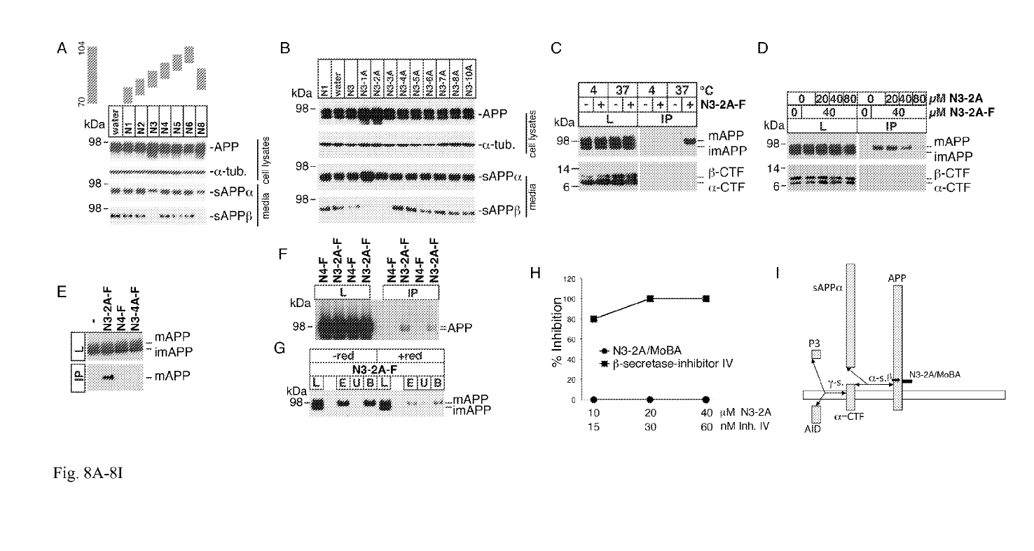

BRI2-derived peptides and APP processing. APP-transfected HEK293 cells were incubated with the indicated peptides for 8 hrs. Peptides were used at either 25 μM (FIG. 8A) or 5 μM (FIG. 8B) concentration.

Precipitation with FLAG-peptides. To prepare brain cells, mouse brains were washed in PBS and minced in dissociation buffer. After sedimentation and filtration, dissociated cells were cultured in Neurobasal media. Cells incubated with Flagged-peptides were lysed and precipitated with Flag-M2-agarose-beads as described (Matsuda et al, 2005). Bound proteins were eluted with 100 μg/ml of FLAG peptide.

Surface biotinylation. HeLa/APP cells were surface biotinylated with sulfo-NHS-SS-biotin and treated with reducing reagent as described (Matsuda et al, 2011a).

Electrophysiological, behavioral and statistical analysis. LTP and NOR were performed as previously described (Bevins & Besheer, 2006; Tamayev et al, 2010b). All data are shown as mean±s.e.m. Statistical tests included two-way ANOVA for repeated measures and t-test when appropriate.

### Experimental Results

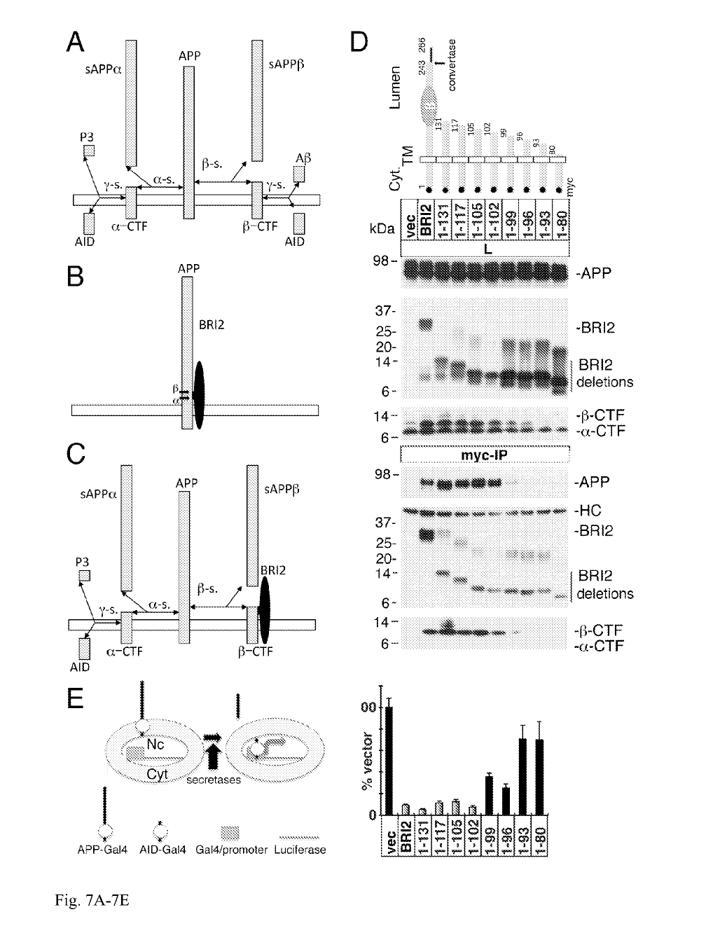

The BRI2 domain that binds APP and inhibits APP processing maps to amino acids 74-102 (see SEQ ID NO:1). To test if the loss of BRI2 in FDD impairs memory via toxic APP metabolites resulting from processing, BRI2-derived peptides were searched for that replicate the inhibitory function of BRI2 on APP-cleavage. BRI2 interacts with mature APP and β-CTF, and increases the levels of β-CTF by inhibiting its γ-cleavage (Matsuda et al, 2005; Matsuda et al, 2008) (FIG. 7B, C). The inhibitory domain was previously mapped to the extracellular region of BRI2 (SEQ ID NO:1) (amino acids 74-131) (Matsuda et al, 2005). To define it further, HeLa cells were co-transfected with APP (HeLa-APP) and myc-tagged BRI2 fragments progressively deleted from the COOH-terminus (FIG. 7D). APP and BRI2 constructs were expressed at similar levels (FIG. 7D). Binding to APP/β-CTF and β-CTF accumulation were progressively abolished particularly between positions 102 to 93 (FIG. 7D). To corroborate these effects on APP processing, BRI2 constructs were co-expressed with an APP-Gal4 fusion construct and a luciferase-reporter under the control of a Gal4-dependent promoter. APPGal4 is a fusion of the yeast transcription factor Gal4 to the cytoplasmic domain of APP. Cleavage of APPGal4 releases the APP intracellular domain (AID)-Gal4 fusion-protein that drives luciferase expression (Gianni et al, 2003) (FIG. 7E). BRI2 blocked most AID-Gal4 release-dependent luciferase activation, but C-terminal deletion again from position 102 to 93 progressively lost this inhibitory activity (FIG. 7E). Thus the functional domain of BRI2 mapped from amino acids 74-102.

A BRI2-derived peptide binds APP and inhibits β-cleavage of APP. It was tested whether peptides spanning this domain duplicated BRI2's function. Two overlapping peptides N3 (SEQ ID NO:4) and N8 (SEQ ID NO:9) strongly reduced β-cleavage and moderately decreased α-processing of APP (FIG. 8A). Mutagenesis of N3 showed that replacing any of amino acid residues 3 to 10 with alanines reduced the inhibitory activity of N3 on β-cleavage of APP, showing the functional importance of these residues. However, replacing either the first or second residue with an alanine (N3-1A/N3-2A) (SEQ ID NO:12 and SEQ ID NO:5, respectively) actually resulted in a stronger inhibitor of APP processing by β-secretase (FIG. 8B). Next it was examined if N3-2A (SEQ ID NO:5) binds APP. HeLa-APP cells were cultured with or without N3-2A fused to a C-terminal Flag epitope (N3-2A-F). After 2 hrs of incubation, cell lysates were precipitated with α-Flag-agarose-beads and co-precipitated molecules were eluted with a Flag-peptide. Like BRI2 N3-2A-F binds mature APP (FIG. 8C). Specificity of this interaction was confirmed by showing that untagged N3-2A could compete for binding to APP (FIG. 8D), and that peptides that do not inhibit APP processing (N4-F or the single amino acid N3 mutant N3-4A-F, FIG. 8A, B), did not bind APP (FIG. 8E). N3-2A-F also binds endogenous APP (FIG. 8F).

N3-2A-F/APP complexes were detected only in metabolically active cells (FIG. 8C). To determine how cell metabolism influences formation of N3-2A/APP complexes, HeLa-APP cells were surface-biotinylated, and cultured with N3-2A-F. After incubation, half of the cells were treated with a reducing reagent, which removes biotin from plasma membrane but not from internalized proteins (+red). N3-2A-F/APP complexes were isolated and further precipitated with streptavidin-beads. In the non-reduced (−red) sample most of APP bound to N3-2A-F was biotinylated (FIG. 8G), suggesting that N3-2A-F binds APP on the cell surface. In the reduced sample, N3-2A-F/APP complexes were found both in intracellular compartments (biotinylated APP) and on the plasma membrane (non-biotinylated APP) (FIG. 8G), supporting the hypothesis that part of plasma membrane N3-2A-F/APP complexes are internalized.

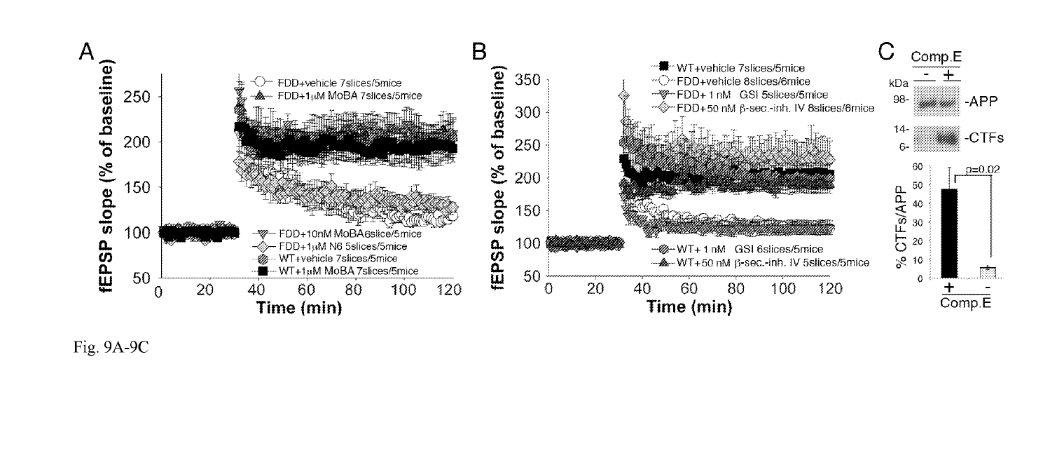

BRI2 binds the region of APP comprising the β-cleavage site, thereby blocking access of this protease to APP, while β-secretase is still active on other substrates. N3-2A did not inhibit the activity of purified β-secretase, while the well-characterized β-secretase-inhibitor IV did (FIG. 8H), indicating that N3-2A has a mechanism of action similar to BRI2 and blocks β-cleavage of APP but not β-secretase. Thus, herein peptide N3-2A is also referred to as Modulator of β-cleavage of APP (MoBA). It should be noted that unlike full-length BRI2, N3-2A/MoBA does not bind β-CTF (FIG. 8C, D) and that at 5 μM concentration this peptide did not overtly inhibit α-processing of APP (FIG. 8B). These data suggest that N3-2A/MoBA interferes with processing of APP by β-secretase and does not modulate γ-cleavage of β-CTF (FIG. 8I) Inhibiting β-, but not γ-, cleavage of APP inhibitor rescue the LTP deficit of FDDKI mice. Long-term potentiation (LTP), a synaptic plasticity phenomenon that underlies memory, is defective in the hippocampal Schaeffer collateral pathway of FDDKI mice. To examine the effect of MoBA on LTP, hippocampal slices were perfused with MoBA for 60 min before inducing LTP. Both at 1 μM or 10 nM concentrations MoBA reversed the LTP deficit of FDDKI samples and did not alter LTP in WT mice (FIG. 9A). N6, which does not inhibit APP processing (FIG. 9A), did not rescue LTP of FDDKI mice (FIG. 9A). Given that β-secretase-inhibitor IV acts similarly to MoBA (FIG. 9B), it is reasonable to conclude that MoBA ameliorates LTP of FDDKI mice by inhibiting β-cleavage of APP and not by unrelated mechanisms Inhibition of β-cleavage of APP could rescue LTP preventing Aβ production, which is considered the primary mediator of synaptic abnormalities in AD.

However, inhibition of Aβ production using the γ-secretase inhibitor (GSI) compound-E did not ameliorate LTP of FDDKI samples (FIG. 9B, C). These findings indicate that β-cleavage of APP during LTP prompts the synaptic plasticity deficits of FDDKI mice and suggest that de novo produced sAPPβ and/or β-CTF and not Aβ, are the synapto-toxic APP species.

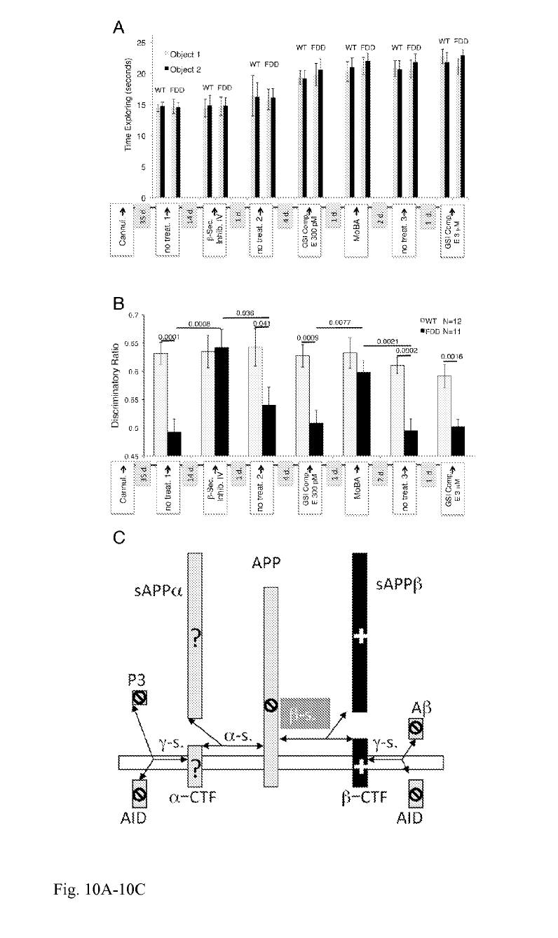

Inhibiting β-, but not γ-, cleavage of APP inhibitor rescue the memory deficit of FDDKI mice. The role of APP processing in the aging-dependent memory deficits of FDDKI mice was next tested (Tamayev et al, 2010b). Novel object recognition (NOR) is a non-aversive memory test that relies on the mouse's natural exploratory behavior. During training, 9-month-old FDDKI and WT mice spent the same amount of time exploring two identical objects (FIG. 10A). The following day, WT mice preferentially explored a novel object that replaced one of the two old objects; conversely FDDKI mice spent the same amount of time exploring the two objects as if they were both novel to them, showing that they had no memory of the objects from the previous day (FIG. 10B). These mice were injected in the lateral ventricle with β-secretase-inhibitor IV and tested again. Treated FDDKI mice spent significantly more time exploring the novel object just as β-secretase inhibitor IV-treated controls (FIG. 10B). A new NOR test showed that, without treatment, FDDKI mice relapsed into amnesia (FIG. 10B), demonstrating that the therapeutic effect of β-secretase inhibition is reversible and short-lived. Next, the behavioral outcome of γ-secretase inhibition was analyzed. The GSI neither improved memory of FDDKI mice nor altered performance of WT animals (FIG. 10B). Then the therapeutic potential of MoBA was assessed in vivo. MoBA significantly improved memory in FDDKI mice and like for β-secretase-inhibitor IV, the therapeutic effect of MoBA was transitory (FIG. 10B). To exclude that compound-E was ineffective due to low dosage, mice were next treated with a ten-fold higher GSI dose. Even this higher dosage did not correct the memory deficit of Danish mice, and GSI-treated WT mice showed a trend, though not statistically significant, toward memory impairment (FIG. 10B). Thus, consistent with the LTP data, β-secretase-inhibitor IV and MoBA rescued, albeit temporarily, the memory deficit of FDDKI mice, while the GSI did not.

### Discussion

The findings here demonstrate that the synaptic plasticity and memory deficits in FDD are mediated through production of sAPPβ and/or β-CTF during LTP and memory acquisition. In addition, they indicate that metabolites derived from γ-cleavage of APP, such as Aβ, P3 and AID/AICD, are not involved in these pathogenic processes (FIG. 10C). Interestingly, it has been suggested that an APP fragment derived from sAPPβ might contribute to AD pathogenesis acting via DR6 (Nikolaev et al, 2009). FDDKI mice are genetically congruous to the human disease, suggesting that the mechanisms underlying synaptic and memory impairments in FDDKI mice faithfully reproduce the pathogenesis of FDD. The inference that Aβ does not cause synaptic and memory dysfunction in FDDKI mice is at odds with the belief that Aβ is the primary mediator of AD-related dementias. Perhaps, FDDKI mice model early pathogenic events preceding amyloid lesions and tauopathy leading to memory loss in human dementia, while Aβ might play a role in later disease stages. It is also possible that the pathophysiology of FDD and AD are distinct and that Aβ is the primary cause of AD but not FDD. However, several analogies exist between FDD and FAD. These two familial dementias have common pathological and clinical presentation. Indeed, FDD presents all the hallmarks of AD. Additionally, FDD and most FAD cases are caused by loss of function mutations of genes that regulate APP processing [BRI2/ITM2b (Tamayev et al, 2010a; Tamayev et al, 2010b) and PSEN1/PSEN2 (DeStrooper, 2007; Saura et al, 2004; Shen & Kelleher, 2007; Zhang et al, 2009)]. The clinical and genetic similarities between FDD and FAD strongly argue that they share common pathogenic mechanisms. Overall, this shows a novel therapeutic approach to reduce sAPPβ/β-CTF levels, and suggest that targeting Aβ production and/or clearance is ineffective or, perhaps, detrimental. Since β-secretase has important biological functions (Hu et al, 2006; Hu et al, 2010; Kim et al, 2007; Willem et al, 2006) the use of a β-secretase inhibitor may produce adverse toxic effects, which would be avoided using compounds like MoBA or compounds with a MoBA-like activity.

### REFERENCES

Akpan N, Serrano-Saiz E, Zacharia B E, Otten M L, Ducruet A F, Snipas S J, Liu W, Velloza J, Cohen G,
Sosunov S A, Frey W H, 2nd, Salvesen G S, Connolly E S, Jr., Troy C M (2011) Intranasal delivery of caspase-9 inhibitor reduces caspase-6-dependent axon/neuron loss and improves neurological function after stroke. J Neurosci 31(24): 8894-8904
Bertram L, Lill C M, Tanzi R E (2010) The genetics of Alzheimer disease: back to the future. Neuron 68(2): 270-281
Bertrand E, Brouillet E, Caille I, Bouillot C, Cole G M, Prochiantz A, Allinquant B (2001) A short cytoplasmic domain of the amyloid precursor protein induces apoptosis in vitro and in vivo. Mol Cell Neurosci 18(5): 503-511
Bevins R A, Besheer J (2006) Object recognition in rats and mice: a one-trial non-matching-to-sample learning task to study ‘recognition memory’. Nat Protoc 1(3): 1306-1311
Choi S I, Vidal R, Frangione B, Levy E (2004) Axonal transport of British and Danish amyloid peptides via secretory vesicles. FASEB J 18(2): 373-375
Cole S L, Vassar R (2007) The Alzheimer's disease beta-secretase enzyme, BACE1. Mol Neurodegener 2: 22
D'Amelio M, Cavallucci V, Middei S, Marchetti C, Pacioni S, Ferri A, Diamantini A, De Zio D, Carrara P, Battistini L, Moreno S, Bacci A, Ammassari-Teule M, Marie H, Cecconi F (2010) Caspase-3 triggers early synaptic dysfunction in a mouse model of Alzheimer's disease. Nat Neurosci 14(1): 69-76
De Strooper B (2007) Loss-of-function presenilin mutations in Alzheimer disease. Talking Point on the role of presenilin mutations in Alzheimer disease. EMBO Rep 8(2): 141-146 De Strooper B, Vassar R, Golde T (2010) The secretases: enzymes with therapeutic potential in Alzheimer disease. Nat Rev Neurol 6(2): 99-107
Eckelman B P, Salvesen G S, Scott F L (2006) Human inhibitor of apoptosis proteins: why XIAP is the black sheep of the family. EMBO Rep 7(10): 988-994
Fotinopoulou A, Tsachaki M, Vlavaki M, Poulopoulos A, Rostagno A, Frangione B, Ghiso J, Efthimiopoulos S (2005) BRI2 interacts with amyloid precursor protein (APP) and regulates amyloid beta (Abeta) production. J Biol Chem 280(35): 30768-30772
Galluzzi L, Joza N, Tasdemir E, Maiuri M C, Hengartner M, Abrams J M, Tavernarakis N, Penninger J, Madeo F, Kroemer G (2008) No death without life: vital functions of apoptotic effectors. Cell Death Differ 15(7): 1113-1123
Ganjei J K (2010) Targeting amyloid precursor protein secretases: Alzheimer's disease and beyond. Drug News Perspect 23(9): 573-584
Garcia-Calvo M, Peterson E P, Leiting B, Ruel R, Nicholson D W, Thornberry N A (1998) Inhibition of human caspases by peptide-based and macromolecular inhibitors. J Biol Chem 273(49): 32608-32613
Gianni D, Zambrano N, Bimonte M, Minopoli G, Mercken L, Talamo F, Scaloni A, Russo T (2003) Platelet derived growth factor induces the beta-gamma-secretase-mediated cleavage of Alzheimer's amyloid precursor protein through a Src-Rac-dependent pathway. J Biol Chem 278(11): 9290-9297
Giliberto L, Matsuda S, Vidal R, D'Adamio L (2009) Generation and Initial Characterization of FDD Knock In Mice. PLoS One 4(11): e7900
Hardy J, Selkoe D J (2002) The amyloid hypothesis of Alzheimer's disease: progress and problems on the road to therapeutics. Science 297(5580): 353-356
Jo J, Whitcomb D J, Olsen K M, Kerrigan T L, Lo S C, Bru-Mercier G, Dickinson B, Scullion S, Sheng M, Collingridge G, Cho K (2011) Abeta(1-42) inhibition of LTP is mediated by a signaling pathway involving caspase-3, Aktl and GSK-3beta. Nat Neurosci News Perspect 23(9): 573-584
Jucker M (2010) The benefits and limitations of animal models for translational research in neurodegenerative diseases. Nat Med 16(11): 1210-1214
Li Z, Jo J, Jia J M, Lo S C, Whitcomb D J, Jiao S, Cho K, Sheng M Caspase-3 activation via mitochondria is required for long-term depression and AMPA receptor internalization. Cell 141(5): 859-871
Lu D C, Rabizadeh S, Chandra S, Shayya R F, Ellerby L M, Ye X, Salvesen G S, Koo E H, Bredesen D E (2000) A second cytotoxic proteolytic peptide derived from amyloid beta-protein precursor. Nat Med 6(4): 397-404
Madeira A, Pommet J M, Prochiantz A, Allinquant B (2005) SET protein (TAF1beta, 12PP2A) is involved in neuronal apoptosis induced by an amyloid precursor protein cytoplasmic subdomain. FASEB J19(13): 1905-1907
McStay G P, Salvesen G S, Green D R (2008) Overlapping cleavage motif selectivity of caspases: implications for analysis of apoptotic pathways. Cell Death Differ 15(2): 322-331
Morrissette D A, Parachikova A, Green K N, LaFerla F M (2009) Relevance of transgenic mouse models to human Alzheimer disease. J Biol Chem 284(10): 6033-6037
Nikolaev A, McLaughlin T, O'Leary D D, Tessier-Lavigne M (2009) APP binds D R6 to trigger axon pruning and neuron death via distinct caspases. Nature 457(7232): 981-989 Passer B, Pellegrini L, Russo C, Siegel R M, Lenardo M J, Schettini G, Bachmann M, Tabaton M, D'Adamio L (2000) Generation of an apoptotic intracellular peptide by gamma-secretase cleavage of Alzheimer's amyloid beta protein precursor. J Alzheimers Dis 2(3-4): 289-301
St George-Hyslop P H, Petit A (2005) Molecular biology and genetics of Alzheimer's disease. C R Biol 328(2): 119-130
Tamayev R, Matsuda S, Giliberto L, Arancio O, D'Adamio L (2011) APP heterozygosity averts memory deficit in knockin mice expressing the Danish dementia BRI2 mutant. EMBO J30(12): 2501-2509
Vidal R, Revesz T, Rostagno A, Kim E, Holton J L, Bek T, Bojsen-Moller M, Braendgaard H, Plant G, Ghiso J, Frangione B (2000) A decamer duplication in the 3′ region of the BRI gene originates an amyloid peptide that is associated with dementia in a Danish kindred. Proc Natl Acad Sci USA 97(9): 4920-4925

1. A method of treating a dementia and/or an impaired cognition in a subject comprising administering to the subject an amount of an inhibitor of caspase-9, of caspase-6 or of caspase-8, sufficient to treat dementia and/or impaired cognition.

2. A method of treating a dementia and/or impaired cognition in a subject comprising administering to the subject an amount of an agent comprising an active fragment of a BRI2 peptide or an active analog of a fragment of a BRI2 peptide sufficient to treat dementia and/or impaired cognition

3. The method of claim 1, wherein the method is for treating a dementia and the dementia is a familial dementia or is caused by Alzheimer's disease.

4. The method of claim 1, wherein the inhibitor of caspase-9, caspase-6 or caspase-8, or the agent, is administered to the subject in a manner effective to cross a central nervous system blood-brain barrier.

5. The method of claim 1, wherein the inhibitor of caspase-9, caspase-6 or caspase-8, or the agent, is administered systemically to the subject.

6. The method of claim 1, wherein the inhibitor of caspase-9, caspase-6 or caspase-8, or the agent, is administered into the central nervous system of the subject.

7. The method of claim 6, wherein the inhibitor of caspase-9, caspase-6 or caspase-8, or the agent, is administered into a cerebral ventricle of the subject.

8. The method of claim 7, wherein the cerebral ventricle is a lateral ventricle.

9. The method of claim 1, wherein the inhibitor of caspase-9, caspase-6 or caspase-8, or the agent, is administered via an implant in the subject.

10. The method of claim 9, wherein the implant is an implanted catheter or pump.

11. The method of claim 9, wherein the implant is implanted into the central nervous system of the subject.

12. The method of claim 1, wherein the inhibitor of caspase-9, caspase-6 or caspase-8, or the agent, is administered continuously to the subject.

13. The method of claim 1, wherein the inhibitor of caspase-9, of caspase-6 or of caspase-8, or the agent, is an siRNA, shRNA, antibody fragment, peptide or small molecule.

14. The method of claim 2, wherein the subject is administered an active fragment of a BRI2 peptide, and wherein the BRI2 peptide comprises consecutive amino acid residues having the sequence set forth in SEQ ID NO:1.

15. The method of claim 2, wherein the subject is administered an agent comprising SEQ ID NO:4, SEQ ID NO:5, SEQ ID NO:9, SEQ ID NO:10 or SEQ ID NO:12.

16. The method of claim 1, wherein the inhibitor is an inhibitor of caspase-9 and comprises XIAP-BIR3 domain (“XBIR3”).

17. The method of claim 1, wherein the inhibitor is an inhibitor of caspase-9 and is XIAP-BIR3 domain disulfide-linked to Penetratin1 (“Pen1-XBIR3”).

18. A method of treating a dementia and/or impaired cognition in a subject comprising administering to the subject an amount of an inhibitor of amino terminal soluble APPβ (sAPPβ) sufficient to treat the dementia and/or impaired cognition.

19-20. (canceled)

21. The method of claim 1, wherein the subject has not suffered a stroke.

22. The method of claim 1, wherein the method is for treating dementia in the subject.

23. The method of claim 1, wherein the method is for treating impaired cognition in the subject.

24-52. (canceled)


SEQUENCE LISTING (18 sequences)

SEQ ID NO: 1 - protein, 266 aa, HOMO SAPIENS
MVKVTFNSALAQKEAKKDEPKSGEEALIIPPDAVAVDCKDPDDVVPVGQRRAWCWCMCFGLAFMLAGVILGGAYLYKYFALQPDDVYYCGIKYIKDDVILNEPSADAPAALYQTIEENIKIFEEEEVEFISVPVPEFADSDPANIVHDFNKKLTAYLDLNLDKCYVIPLNTSIVMPPRNLLELLINIKAGTYLPQSYLIHEHMVITDRIENIDHLGFFIYRLCHDKETYKLQRRETIKGIQKREASNCFAIRHFENKFAVETLICS

SEQ ID NO: 2 - protein, 10 aa, ARTIFICIAL SEQUENCE
  FRAGMENT DIRECTED TO A HOMO SAPIENS PROTEIN
YLYKYFALQP

SEQ ID NO: 3 - protein, 10 aa, ARTIFICIAL SEQUENCE
  FRAGMENT DIRECTED TO A HOMO SAPIENS PROTEIN
FALQPDDVYY

SEQ ID NO: 4 - protein, 10 aa, ARTIFICIAL SEQUENCE
  FRAGMENT DIRECTED TO A HOMO SAPIENS PROTEIN
DDVYYCGLKY

SEQ ID NO: 5 - protein, 10 aa, ARTIFICIAL SEQUENCE
  FRAGMENT DIRECTED TO A HOMO SAPIENS PROTEIN
DAVYYCGLKY

SEQ ID NO: 6 - protein, 10 aa, ARTIFICIAL SEQUENCE
  FRAGMENT DIRECTED TO A HOMO SAPIENS PROTEIN
CGLKYIKDDV

SEQ ID NO: 7 - protein, 10 aa, ARTIFICIAL SEQUENCE
  FRAGMENT DIRECTED TO HOMO SAPIENS PROTEIN
IKDDVILNEP

SEQ ID NO: 8 - protein, 10 aa, ARTIFICIAL SEQUENCE
  FRAGMENT DIRECTED TO HUMAN PROTEIN
ILNEPSADAP

SEQ ID NO: 9 - protein, 14 aa, ARTIFICIAL SEQUENCE
  FRAGMENT DIRECTED TO HUMAN PROTEIN
DDVYYCGLKYIKDD

SEQ ID NO: 10 - protein, 10 aa, ARTIFICIAL SEQUENCE
  FRAGMENT DIRECTED TO HUMAN PROTEIN
DXVYYCGLKY

SEQ ID NO: 11 - protein, 4 aa, ARTIFICIAL SEQUENCE
  CHEMICALLY SYNTHESIZED
LEHD

SEQ ID NO: 12 - protein, 10 aa, ARTIFICIAL SEQUENCE
  FRAGMENT DIRECTED TO HOMO SAPIENS PROTEIN
ADVYYCGLKY

SEQ ID NO: 13 - protein, 4 aa, ARTIFICIAL SEQUENCE
  CHEMICALLY SYNTHESIZED
LEHD

SEQ ID NO: 14 - protein, 4 aa, ARTIFICIAL SEQUENCE
  CHEMICALLY SYNTHESIZED
DEVD

SEQ ID NO: 15 - protein, 116 aa, HOMO SAPIENS
SDAVSSDRNFPNSTNLPRNPSMADYEARIFTFGTWIYSVNKEQLARAGFYALGEGDKVKCFHCGGGLTDWKPSEDPWEQHAKWYPGCKYLLEQKGQEYINNIHLTHSLEECLVRTT

SEQ ID NO: 16 - protein, 497 aa, HOMO SAPIENS
MTFNSFEGSKTCVPADINKEEEFVEEFNRLKTFANFPSGSPVSASTLARAGFLYTGEGDTVRCFSCHAAVDRWQYGDSAVGRHRKVSPNCRFINGFYLENSATQSTNSGIQNGQYKVENYLGSRDHFALDRPSETHADYLLRTGQVVDISDTIYPRNPAMYSEEARLKSFQNWPDYAHLTPRELASAGLYYTGIGDQVQCFCCGGKLKNWEPCDRAWSEHRRHFPNCFFVLGRNLNIRSESDAVSSDRNFPNSTNLPRNPSMADYEARIFTFGTWIYSVNKEQLARAGFYALGEGDKVKCFHCGGGLTDWKPSEDPWEQHAKWYPGCKYLLEQKGQEYINNIHLTHSLEECLVRTTEKTPSLTRRIDDTIFQNPMVQEAIRMGFSFKDIKKIMEEKIQISGSNYKSLEVLVADLVNAQKDSMQDESSQTSLQKEISTEEQLRRLQEEKLCKICMDRNIAIVFVPCGHLVTCKQCAEAVDKCPMCYTVITFKQKIFMS

SEQ ID NO: 17 - protein, 116 aa, HOMO SAPIENS; identical to SEQ ID NO: 15

SEQ ID NO: 18 - protein, 16 aa, DROSOPHILA
RQIKIWFQNRRMKWKK